(19) United States
(12) Patent Application Publication
(10) Pub. No.: US 2019/0110744 A1
(43) Pub. Date: Apr. 18, 2019

(54) INTERCHANGEABLE WEARABLE DEVICE

(71) Applicant: Jiping Zhu, Flushing, NY (US)

(72) Inventor: Jiping Zhu, Flushing, NY (US)

(21) Appl. No.: 15/787,166

(22) Filed: Oct. 18, 2017

(51) Int. Cl. A61B 5/00; A61B 5/0205; A61B 5/0404

(52) U.S. Cl. CPC A61B 5/681; A61B 5/02055; A61B 5/0404; A61B 5/02438; A61B 5/7445; A61B 5/7475; A61B 5/7405

(57) ABSTRACT

Disclosed is a wearable device with different types of interchangeable accessory covers that may be removably attached thereto. The wearable device has a processor and sensor(s) for taking measurements (e.g., for health of a user), as well as buttons on its opposing sides, one of which includes braille thereon for distinguishing purposes. Each of the covers includes a different accessory—e.g., necklace attachment portion, clip, watch lugs, sensors—or combination thereof, enabling different options to a user for wearing the wearable device. In one option, the covers are attached to a back of the wearable device. Each cover is configured to provide accessibility to an electrical connector of the wearable device. In some cases, the covers themselves include an electrical connector portion that is used to establish an electrical connection between the processor of the wearable device and an external device to communicate measurements via the attached interchangeable accessory cover.

### BACKGROUND

### Field

This disclosure relates generally to a wearable device. More particularly, the present disclosure relates to a wearable device and attachments, as well as communication link between the wearable device and other devices.

### Description of the Related Art

Some people wear electronic devices wrapped around their wrist or arm, much like a bracelet. Alternatively, some electronic devices may be carried in a pocket or purse, or worn on another part of a person, e.g., on a piece of clothing. Such electronic devices may include features relating to a watch (i.e., telling time of day), a fitness or an activity tracker (e.g., number of steps walked, sleep pattern, etc.) or other health monitoring devices, including sensors, and are sometimes also referred to as smart watches. Generally, users are able to download, communicate with, and/or transmit data collected by their electronic device to another device, such as a smart phone, a laptop, or computer.

### SUMMARY

According to one aspect of this disclosure, there is provided a wearable device. The wearable device includes a device case that has: at least one measurement device and a processor for processing measurements collected by the measurement device that are enclosed within the case, and a display or screen on a front of the device case for displaying data corresponding to measurements measured by the at least one measurement device. The device case also has a first button provided on one side of the device case, the first button having a raised, tactile portion thereon, and a second button provided on an opposite side of the device case, relative to the first button. The first and second buttons are configured to implement a function or an action via the processor when pressed by a user. The device case also has an electrical connector portion provided on a back of the device case that is used to establish an electrical connection between the processor and an external device such that the processor and the external device are configured for communication and measurements that are collected by the at least one measurement device and processed by the processor are communicated to the external device. The wearable device also includes an interchangeable accessory cover removably attached to the device case. The accessory cover is configured to provide accessibility to the electrical connector portion on the back of the device case when attached thereto.

Another aspect of this disclosure provides a system comprising: a wearable device including: a device case that has: at least one measurement device and a processor for processing measurements collected by the measurement device that are enclosed within the case, and a display or screen on a front of the device case for displaying data corresponding to measurements measured by the at least one measurement device. The device case also has a first button provided on one side of the device case, the first button having a raised, tactile portion thereon, and a second button provided on an opposite side of the device case, relative to the first button. The first and second buttons are configured to implement a function or an action via the processor when pressed by a user. The device case also has an electrical connector portion provided on a back of the device case that is used to establish an electrical connection between the processor and an external device such that the processor and the external device are configured for communication and measurements that are collected by the at least one measurement device and processed by the processor are communicated to the external device. The wearable device also includes a first interchangeable accessory cover and a second interchangeable accessory cover, each adapted to be removably attached to the device case and each of the first and second interchangeable accessory covers are configured to provide accessibility to the electrical connector portion on the back of the device case when attached thereto. The second interchangeable accessory cover has a different configuration than the first interchangeable accessory cover.

Yet another aspect of this disclosure provides an interchangeable accessory cover for removable attachment to a wearable device, the wearable device having: a housing that contains at least one measurement device and a processor for processing measurements collected by the measurement device, and a display or screen on a front of the housing for displaying data corresponding to measurements measured by the at least one measurement device. The accessory cover has a front portion and a back portion, and an electrical connector portion with electrical contact points that are accessible via the back portion upon attachment to the wearable device. The electrical connector portion is configured to establish an electrical connection between the processor of the wearable device and an external device such that the processor and the external device are configured for communication and measurements that are collected by the at least one measurement device and processed by the processor are communicated to the external device via the interchangeable accessory cover.

The forgoing general description of the illustrative implementations and the following detailed description thereof are merely exemplary aspects of the teachings of this disclosure, and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1A is a front side perspective view of a wearable device in accordance with an embodiment of this disclosure;

FIGS. 1B, 1C, 1D, 1E, 1F, and 1G are front, top, bottom, first (left) side, second (right) side, and back views, respectively, of the wearable device of FIG. 1A;

FIGS. 1H, II, and IJ are exploded back side, front side, and second (right) side views, respectively, of the device case and the interchangeable accessory cover of the wearable device of FIG. 1A;

FIG. 1K is a schematic diagram of features contained within the device case of the wearable device of FIG. 1A according to an embodiment;

FIG. 2A is an exploded, back side angled view of a second interchangeable accessory cover and a device case of a wearable device according to another embodiment of this disclosure;

FIGS. 2B, 2C, 2D, 2E, 2F, and 2G illustrates front, top, bottom, first side, second side, and back views, respectively, of the wearable device of FIG. 2A;

FIGS. 2H and 2I are exploded front side and second (right) side views, respectively, of the device case and the interchangeable accessory cover of the wearable device of FIG. 2A;

FIG. 3A is an exploded, back side angled view of a third interchangeable accessory cover and a device case of a wearable device in accordance with another embodiment of this disclosure;

FIGS. 3B, 3C, 3D, 3E, 3F, and 3G illustrates front, top, bottom, first side, second side, and back views, respectively, of the wearable device of FIG. 3A;

FIGS. 3H and 3I are exploded front side and second (right) side views, respectively, of the device case and the interchangeable accessory cover of the wearable device of FIG. 3A;

FIG. 4A is an exploded, back side angled view of a fourth interchangeable accessory and a device case of a wearable device according to yet another embodiment of this disclosure;

FIG. 4B is a front, angled view of the fourth interchangeable accessory of FIG. 4A;

FIGS. 4C, 4D, 4E and 4F illustrate front, second (right) side, front perspective, and back perspective views, respectively, of the wearable device of FIG. 4A;

FIG. 4G is an exploded second (right) side view of a device case and the interchangeable accessory cover of the wearable device of FIG. 4A;

FIG. 5A an exploded, back side angled view of a fifth interchangeable accessory cover and a device case of a wearable device according to still yet another embodiment of this disclosure;

FIG. 5B is an exploded, front side angled view of the fourth interchangeable accessory and device case of FIG. 4A;

FIGS. 5C, 5D, 5E and 5F illustrate front, second (right) side, front perspective, and back perspective views, respectively, of the wearable device of FIG. 5A;

FIG. 5G is an exploded second (right) side view of a device case and the interchangeable accessory cover of the wearable device of FIG. 5A;

FIGS. 6A and 6B illustrates a schematic representation of exploded, right side views of the device case and an interchangeable accessory cover having cooperative threaded attachment mechanisms for attachment, according to embodiments of the present disclosure;

FIG. 7 illustrates a schematic representation of an exploded, right side view of the device case and an interchangeable accessory cover including a snap fit attachment mechanism according to another embodiment of the present disclosure;

FIG. 8 illustrates a schematic representation of an exploded, right side view of a retractable pin attachment mechanism for the device case and an interchangeable accessory cover according to yet another embodiment of the present disclosure;

FIG. 9 illustrates a schematic representation of an exploded, right side view of the device case and an interchangeable accessory cover including clip based attachment mechanism for attachment according to still yet another embodiment of the present disclosure; and

FIGS. 10A and 10B illustrates a schematic representation of exploded, right side views of the device case and a interchangeable accessory cover having magnetic attachments according to additional embodiment of the present disclosure.

### DETAILED DESCRIPTION

The description set forth below in connection with the appended drawings is intended as a description of various embodiments of the disclosed subject matter and is not necessarily intended to represent the only embodiment(s). In certain instances, the description includes specific details for the purpose of providing an understanding of the disclosed embodiment(s). However, it will be apparent to those skilled in the art that the disclosed embodiment(s) may be practiced without those specific details. In some instances, well-known structures and components may be shown in block diagram form in order to avoid obscuring the concepts of the disclosed subject matter.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation, or any requirement that each number must be included.

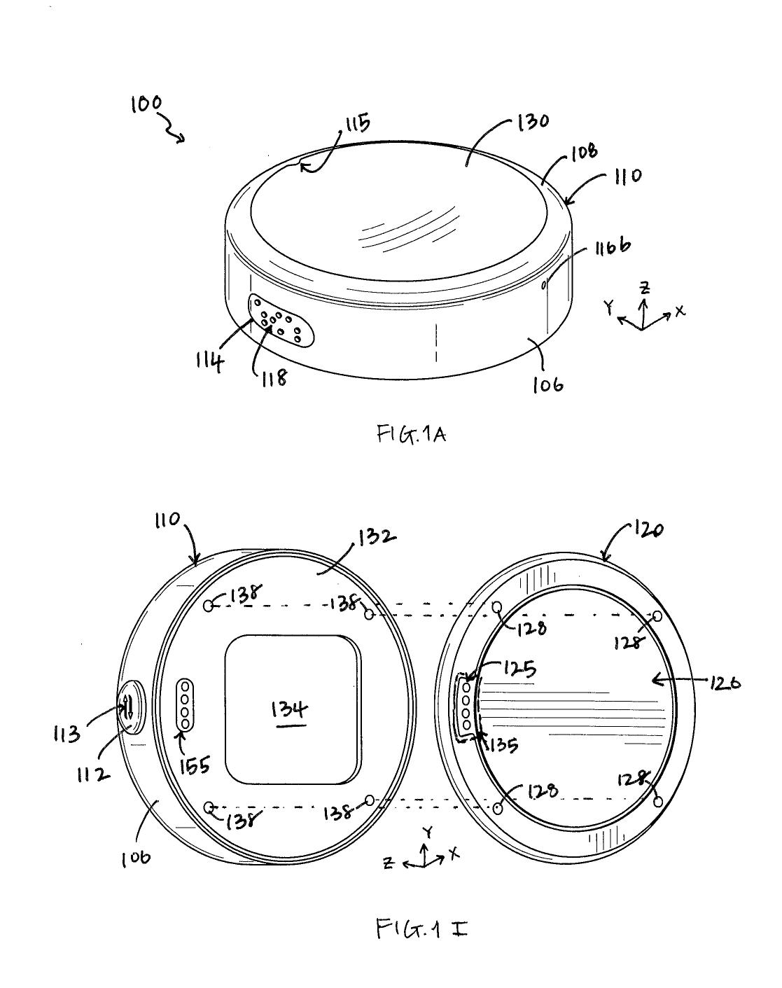

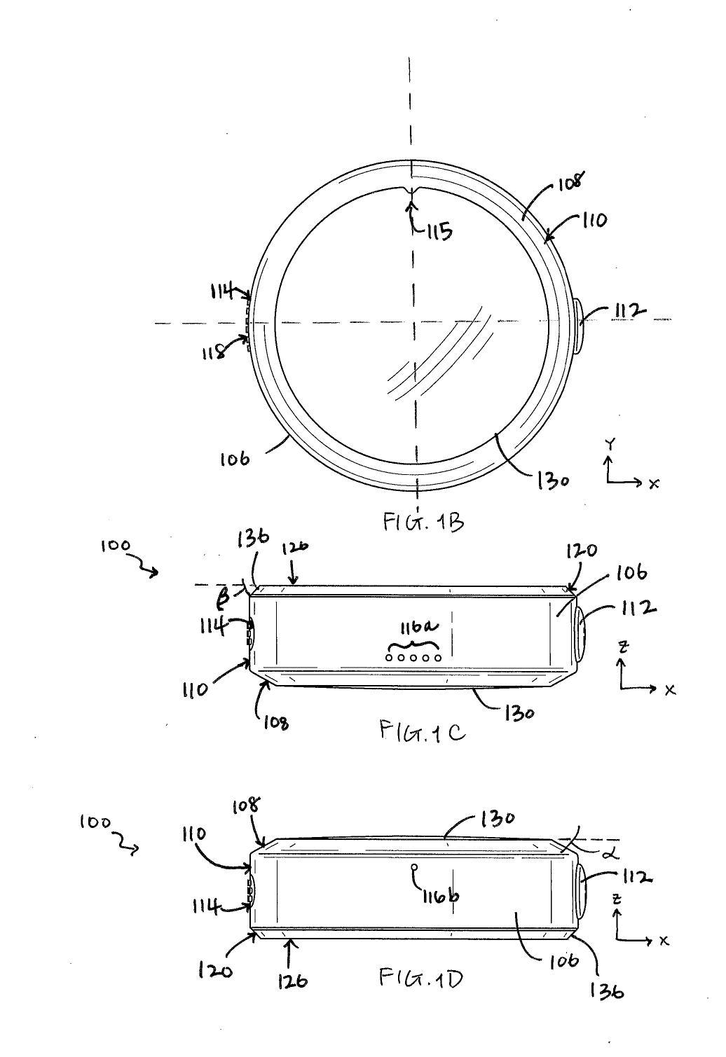

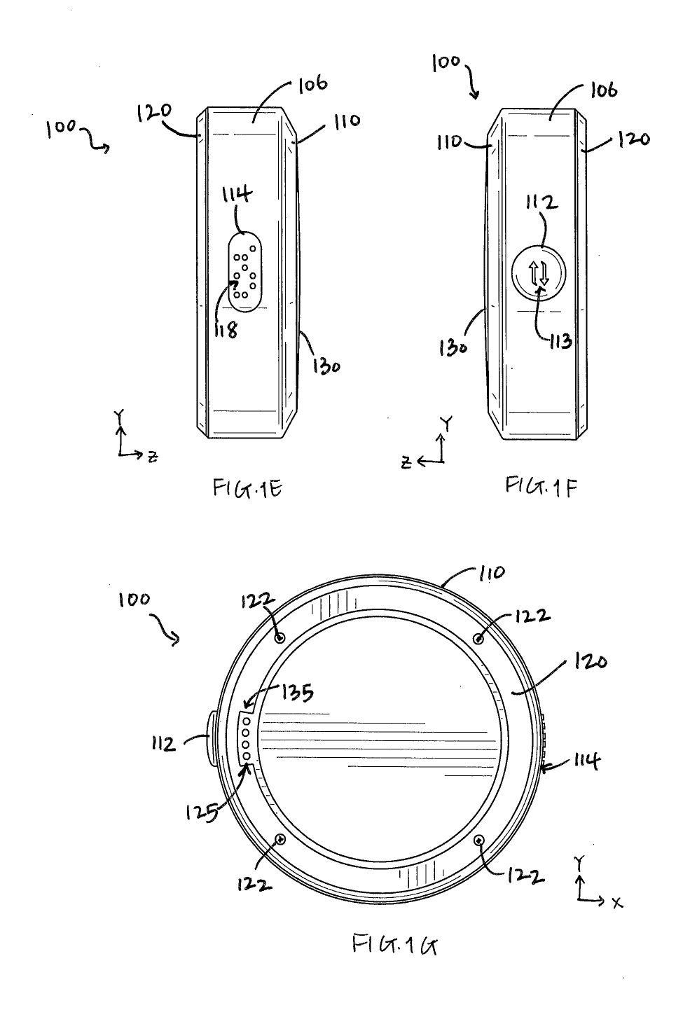

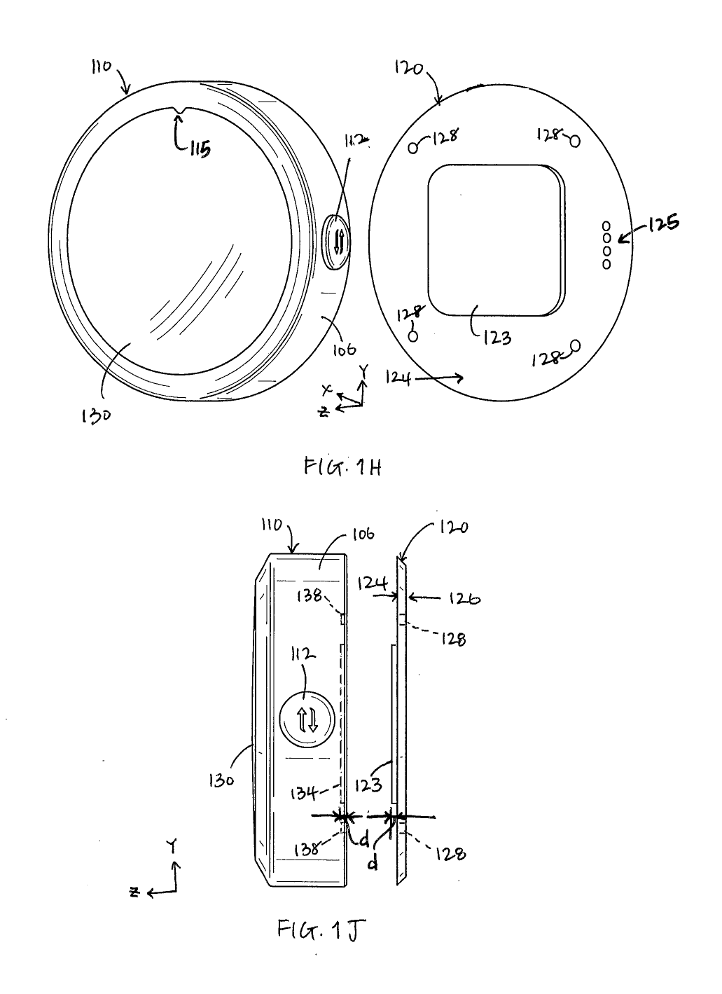

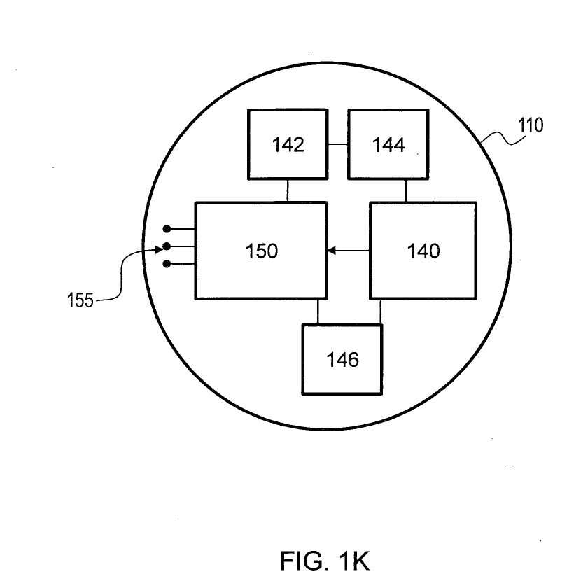

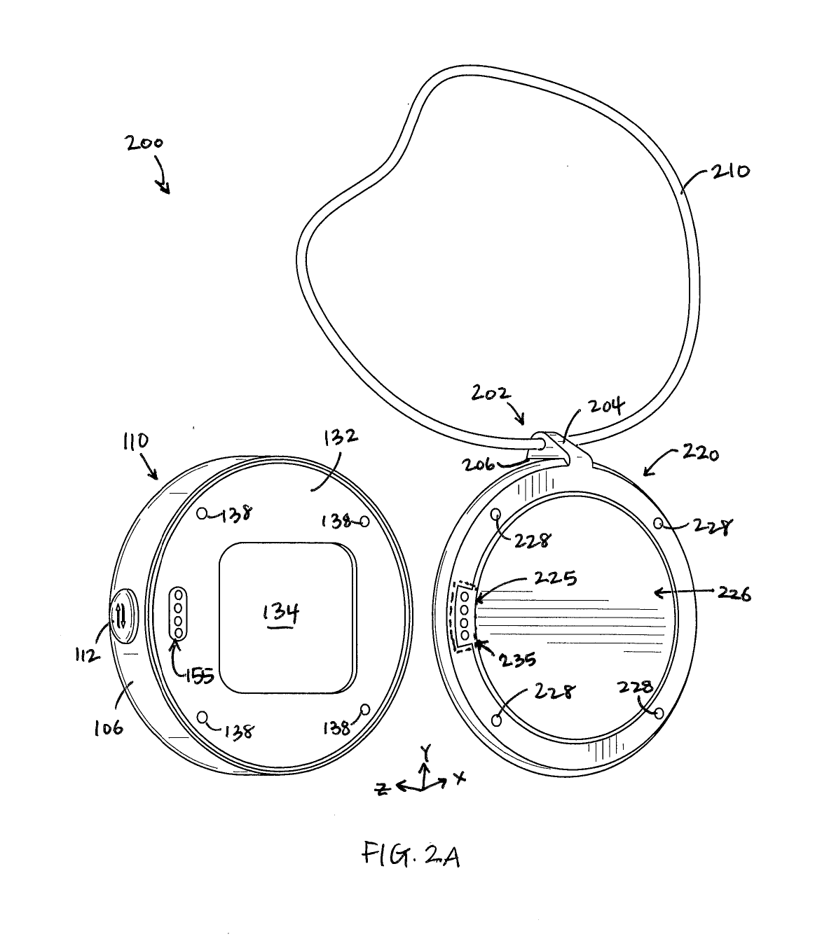

FIGS. 1A-1J illustrate multiple views of parts of a wearable device 100 according to an embodiment of this disclosure. In some instances, wearable device 100 may be referred to as a “smart case” or “smart watch” herein. For example, in accordance with an embodiment, the wearable device 100 is designed for measurement, diagnostic, and/or monitoring purposes with regards to lifestyle, activities, and health of a user (e.g., number of steps taken, heartrate and/or cardiac readings, location (GPS)). The wearable device 100 may be used to keep track of physical activities of a user or wearer such as a number of steps that a user has walked via, e.g., detected motion via an accelerometer, and then send such activity data to an application that is associated with or provided on an external device (e.g., an application installed on a user's smart phone or mobile phone, or a computer, or a server) so that the user and/or other health professional is able to keep track of a level of activity each day for that user. The wearable device 100 may also detect and track a user's vital signs and/or physical location, e.g., via a positioning service, GPS, etc., in accordance with an embodiment. As another example, such as for in the elderly care industry, in addition to health related measurements, the wearable device 100 may include communication features, including the ability for a user to initiate an SOS or emergency call in an emergency situation and/or listen to and/or speak to a representative provided at a remote location, in accordance with an embodiment.

Processing features of a device case of the wearable device 100 are generally discussed throughout this disclosure and schematically shown with reference to FIG. 1K. The schematic drawing is representative only and not intended to be limiting with regards to the illustrated features. The wearable device includes a device case 110 that includes a housing which contains or encloses one or more measurement device(s) and/or one or more sensor(s) therein, generally represented as 140, a battery 144, a memory 146, and a processor 150 or processing circuitry for receiving and processing measurements/sensed data collected by the measurement device(s)/sensor(s) 140. The processing circuitry 150 may be configured to monitor a wearer's health, display time, alert signals or other monitoring functionality. Examples of possible processing circuitry and related functions provided in the device case 110 and wearable device 100 may be found in U.S. application Ser. No. 15/299,025, filed on Oct. 20, 2016, and Ser. No. 15/298,947, filed on Oct. 20, 2016, both of which are incorporated by reference herein in their entireties. The one or more sensors 140 may be physiological sensors such as a heart rate sensor, a blood sugar level sensor, a temperature sensor, a skin conductivity sensor, and/or an electrocardiograph sensor configured to measure the wearer's vitals, as well as step counting sensors (accelerometers), location sensing device (GPS), and the like. In an embodiment, one or more of sensors may also be placed on the interchangeable accessory cover and configured to communicate with the processing circuitry 150 via electrical connectors. In an embodiment, one or more of the sensors 140 and/or sensors on the accessory cover(s) may communicate wirelessly with the processing circuitry 150. The housing of the device case 110 further includes an audio device 142 or speaker therein that is configured to emit sound(s) or signal(s), e.g., as prompted by processor 150, to a user of the wearable device 110. As generally understood by one of ordinary skill in the art, sounds or signals that are emitted by the wearable device 100 may include an alarm (e.g., beeping) to alert the user of the wearable device 100, clicks or positive/negative sounds for providing feedback to the user, and/or spoken words, phrases, and/or a voice of a remote party that utilizes the device's communication features, e.g., via an SOS or emergency call.

Referring now specifically to FIGS. 1A-1J, the wearable device 100 has a main body portion or housing—referred to herethroughout as a “device case” 110—and one or more accessories or accessory covers that attach to it in some manner, so that the wearable device 100 may be worn on different parts of a user's body. The housing of the device case 110 has a front, a top, a bottom, a first (left) side, a second (right) side, and a back, respectively, shown in FIGS. 1B through 1F, and FIG. 1I, along with an interchangeable accessory cover 120 attached thereto in the form of a back cover. In accordance with an embodiment, the housing of the device case 110 may be generally circular or round and have a perimeter or circumferential edge 106 extending between its front face and a back plate 132 (see FIG. 1l) that defines its side(s). As generally understood in the art, the housing includes a hollow receiving area (not shown) (e.g., between the front face and the back plate 132) for receiving and containing electronic parts, such as the sensors/measurement devices 140, audio device 142, and processor 150, for the wearable device 100.

In addition to containing the above-described sensors 140 and processor 150 therein, the device case 110 has a display 130 or screen on or across the front or face of the case 110, e.g., see FIG. 1B. The display 130 or screen is configured to display data corresponding to measurements measured by the at least one measurement device 140, user information, options and/or settings, and the like. For example, the data that is displayed may be direct measurement data, e.g., a number of steps, or indirect/calculated data, that is processed by the processor 150. The display 130 may also be used to show a user a variety of options and menu items for customization and/or relating to the user themselves, which is generally known in the art and thus not further described here. The display 130 includes a glass or plastic portion, for example. The front surface or face of the display 130 may be substantially flat or slightly rounded. The type of display or screen is not limited—the display or screen may be a LCD or an LED type of display, for example. In an embodiment, the display 130 has an outer perimeter or an edge that is substantially circular in shape, when viewing from the front, i.e., in Z direction (see, e.g., FIG. 1B). However, the display 130 may also be oval, rectangular, or another geometric shape. In one embodiment, the shapes of the housing and display 130 are similar, e.g., both are substantially circular. In another embodiment, the shapes of the housing and display 130 are different, e.g., one is substantially ovular, while another is substantially circular.

Surrounding the display 130 is a front edge 108 of the device case 110. The front edge 108 may include a bezel or groove for holding the glass or plastic portion of the display 130 in position. As seen in FIGS. 1C and 1D, the front edge 108 may be a beveled front edge that is provided at an angle α relative to a plane that extends (horizontally) across the display 130. The beveled front edge 108 extends between a perimeter or a circumferential edge of the display 130 and an edge or side surface of the housing of the device case 110. The beveled front edge 108 provides better viewing and generally makes the wearable device 100 appear smaller.

In accordance with an embodiment, an orientation indicator 115 is provided on or in the housing of the device case 110 to indicate a viewing direction or position for viewing the display 130 or screen, e.g., so that a user may recognize a correct orientation of the wearable device 100, even when the display 130 is off. As shown in FIG. 1B, for example, the orientation indicator 115 may be provided on the front of the device case 110 in the form of a small pointed portion extending from the front edge 108 of the housing or case. In an embodiment, the pointed portion of the orientation indicator 115 is positioned over the display 130 or screen on the front of the device case. In an embodiment, the orientation indicator 115 may be located at a 12 o'clock position typically associated with a watch or a clock. In accordance with an embodiment, the orientation indicator 115 may be a structural part that is formed as part of the housing of the device case 110. In one embodiment, the orientation indicator 115 may be continuously formed with the front edge 108, e.g., as a part of the bezel, while an edge of the glass or plastic portion of the display 130 is formed such that it includes a corresponding or complimentary shaped indentation or cutout (e.g., extending into the display 130 from its edge) for receiving the orientation indicator 115 therein (when the device case 110 is assembled). The shape of the orientation indicator 115 that is formed in the front edge 108 or in the housing (as part of its front face) is not intended to be limited to the illustrated pointed portion—it may be angular, triangular, or polygonal in shape or any other geometrical shape, for example, that projects over or into a portion of the display 130.

Alternatively, in another embodiment, the orientation indicator 115 may also or alternatively include an indentation or a marking provided on the front edge 108 of the device case 110 that does not affect or change the edge of the display 130. Alternatively, or in addition, the orientation indicator 115 may be configured to be displayed on the display 130 or screen when the wearable device 100 is turned on.

In an embodiment, the orientation indicator 115 may also indicate a position for aligning the wearable device 100 with electrical connectors of an external device.

As shown in FIG. 1C, one or more speaker holes 116a may be provided on a side of the device case 110, e.g., a top side or top edge of the housing (part of edge 106). The location of the speaker holes 116a is not intended to be limiting; e.g., they may also or alternatively be provided on a bottom edge. In an embodiment, the speaker holes 116a may be provided on a top with respect to a vertical centerline (see FIG. 1B, in the Y-direction) of the device case 110. The speaker holes 116a may be configured to emit sound(s) or signal(s) such as an alert, voice of a remote party, etc., as noted above, from the audio device 142 and/or communication features of the wearable device 100. The holes 116a are designed such that audio may be transmitted at its maximum capacity. The speaker holes 116a may be water resistant.

Another hole 116b or holes may be provided on another side of the device 110, e.g., on a bottom side or bottom edge of the housing (part of edge 106), as shown in FIG. 1D. In one embodiment, hole 116b is a reset access point, wherein a device or tool may be inserted through the hole 116b to press a button or part on an electronic part contained within the housing of device case 110, to reset or restart the electronics and/or display of the wearable device 110. In another embodiment, the hole 116b may be a speaker hole. In yet another embodiment, the hole 116b is simply included as part of the housing for air flow.

In accordance with one embodiment, the speaker holes 116a are provided on a first side (e.g., top) of the wearable device 100 and the hole 116 is provided on a second side that is opposite to the first side (e.g., bottom). In an embodiment, the speaker holes 116a and hole 116b may be placed on the edge 106 relative to the vertical centerline (see FIG. 1B) of the device case 110.

The device case 110 also has a first button 114 provided on one side of the case, e.g., a left side of the case as shown in FIG. 1E, and a second button 116 provided on an opposite side, e.g., a right side, of the case (opposite relative to the side that button 114 is provided on), as shown in FIG. 1F. In an embodiment, the first button 114 and the second button 112 may be located on the edge 106 and aligned along a horizontal centerline (see FIG. 1B, in the X-direction) of the device case 110. The buttons 114, 112 may be pressed to move (e.g., inwardly) relative to the edge 106 or side. The first and second buttons 114, 112 may be configured to implement a function or an action via the processor 150 when they are pressed (e.g., inwardly) by a user.

In one embodiment, the first button 114 may be ovular in shape, for example, as shown in FIG. 1E. However, the button 114 may be circular, rectangular, or other shapes. In an embodiment, the first button 114 has a raised, tactile portion 118 or textured portion (e.g., bumps) on its surface. This textured or tactile portion 118 enables a user to easily distinguish the first button 114 from the second button 112 on the opposite side of the device case 110 based on touch (using a finger). In one embodiment, the raised, tactile portion 118 is in the form of braille. In accordance with one embodiment, button 114 is designed as an emergency button, e.g., used to make an SOS call via the wearable device 100 to another party. For example, in the elderly care industry, when the user feels that he/she is in an emergency situation, such as a fall or in a health crisis situation, the user may physically activate the button 112 on the device 100 to trigger a signal to be sent from the device (e.g., via its processor 150 and/or communication features) to an emergency handling service, service center, or designated party. In a case where braille is provided as the tactile portion 118 on button 114, the braille may read “SOS”, for example. The user may receive commands, updates, alerts, or instructions via the audio device 142 emitting sound(s) through the speaker holes 116, for example.

In one embodiment, the second button 112 may be circular in shape, for example, as shown in FIG. 1F. However, the button 112 may be ovular, rectangular, or other shapes. The button 112 may be in communication with the processing circuitry 150 to allow navigation between menus and/or data displayed via the display/screen 130. Further, in an embodiment, the button 112 may include arrows 113, or other indicators, in or on its surface to visually indicate its purpose and/or navigation directions. The button 112 may also trigger other functions related to the display 130, including a rotation or flipping operation of an object displayed on the screen.

In accordance with one embodiment, the first button 114 of the wearable device 100 is ovular in shape and the second button 112 is circular in shape.

A back of the device case 110, i.e., the face that is opposite to the display 130, includes the back plate 132 which is connected to the edge 106 to contain its electronic components (e.g., see FIG. 1K) therein. The device case 110 also has an electrical connector portion 155 provided on its back plate 132, as shown in FIG. 1I, for example, and generally represented in FIG. 1K. In accordance with an embodiment, the electrical connector portion 155 is used to establish an electrical connection between the processor 150 within the device case 110 and an external device (e.g., a charging station or a base station, which are not shown) such that measurements that are collected by the at least one sensor/measurement device 140 and processed by the processor 150 are communicated to the external device. Generally, the external device is designed to not only charge the wearable device 100, but to also communicate with the wearable device 100 (e.g., processor 150) to receive and send data and information. The electrical connector portion 155 may include one or more contact points or electrical connectors associated therewith. In an embodiment, such an electrical connector portion 155 may be formed from wires that extend from the processor and into holes of the connector portion such that an end of each wire is accessible. In the exemplary illustrated embodiments of the figures, the back plate 132 includes four electrical wire ends. Each of the wires of the connector portion 155 may have separate functions; for example, one wire of the electrical connector portion 155 may be used for charging the wearable device 100, and other (e.g., three others, as shown here) electrical connectors of the electrical connector (125) may be used for establishing a communication link between the wearable device 100 (e.g., a processor therein) and an external device. As such, the electrical connector portion 155 may be used to charge the wearable device 100 as well as transfer data or health related information collected by the wearable device 100 to the external device.

In an embodiment, the electrical connectors may be made of electrically conducting material (e.g., copper, aluminum, or other metal). The electrical connectors may take several forms and shapes, for example, the electrical connector may be in the form of wires, and/or a strip, may be flexible and/or may be etched on a surface of the device case 100 (and/or the interchangeable accessory cover, described later). In an embodiment, the electrical connectors may be contact points or a contact surface. In an embodiment, the electrical connectors may include wire with terminals having flat end connected by a wire or a strip. In another embodiment, the terminals may be connected to contact pins, for example, by clamping crimping, soldering, screw termination, etc. In yet another embodiment, the electrical connectors may be ring-shaped or a hollow cylinder such that an electrical contact may be established via a pin or wire type of electrical contacts of the interchangeable accessory cover.

The wearable device 100 also includes an interchangeable accessory cover 120 removably attached to the device case 110. One exemplary embodiment of the interchangeable accessory cover 120 is best seen in FIGS. 1G-IJ. Among other things, the accessory cover 120 is configured to provide accessibility to the electrical connector portion 155 on the back of the device case 110, when the accessory cover 120 is attached thereto. As described later, the accessibility to electrical connector portion 155 provided by the accessory cover 120 may be via an opening, a hole, a cutout portion, or a shape of the cover itself, or via an electrical connector portion 125 provided on the accessory cover 120. In one embodiment, the device case 110 and the accessory cover 120 may be communicably coupled to each other such that the wearable device 100 may establish an electrical connection as well as communication link with an external device when the cover 120 is placed into electrical contact with the external device.

In accordance with embodiments herein, the interchangeable accessory cover 120 is configured to cover at least a portion of the back of the device case 110 when attached to the device case. In some cases, the accessory cover 120 is configured to cover an entire back of the device case 110. Generally, the interchangeable accessory cover 120 is shown and described herein as being removably attached to a back of the device case 110 (specifically, back plate 132); however, the illustrated exemplary embodiments should not limit the interchangeable accessory cover 120 from including or having any attachment or configuration that attaches to a portion of the sides or edge 106, in addition or alternatively to the back of the device case 110. For example, the cover 120 may be designed to cover or surround at least part of the back, while using a securement device or mechanism on or around an edge 106 or side of the device case 110.

As shown in FIGS. 1A-1J, for example, the interchangeable accessory cover 120 may be provided in the form of a plate with a generally flat outer (back) surface 126 and shaped in the form of a disc, which is similar to the shape of back plate 132 (e.g., both are substantially circular). Thus, when the accessory cover 120 is attached to the device case 110, it forms a wearable device 100 having a puck-like shape that may be easily slipped into a user's pocket. Like the front edge 108 of the device case 110, the accessory cover 120 may include a beveled edge 136 that is provided at an angle 3 (see FIG. 1C) relative to a plane extending across its generally flat, outer surface 126. In one embodiment, as shown in FIGS. 1B-IJ, the accessory cover 120 is similarly shaped to the housing of device case 110—or at least its back plate 132—for example, substantially circular in shape. In accordance with embodiments herein, the device case 110 and the accessory cover 120 may be similarly shaped (circular, oval, rectangular or other polygonal or geometric shape).

In addition to its back portion or outer surface 126 (see FIGS. 1I and 1J), the interchangeable accessory cover 120 has a front portion or inner surface 124 (see FIGS. 1H and 1J). The front portion is configured for placement against a portion of the device case 110; specifically, as shown in FIGS. 1H and 1I, structural features are provided on the cover 120 and device case 110 to assist in alignment and attachment. The inner surface 124 of cover 120 has a projection 123 provided on its inner surface 124 that extends a distance d (see FIG. 1J, in the Z-direction) away from the surface, that is visible when viewed from the X-direction. Back plate 132 of the device case 110 has a recess 134 therein that is complimentary to the shape of the projection 123 (here, as an example, both the projection 123 and recess 134 are shown as being generally rectangular in shape). Accordingly, recess 134 may extend into the housing at distance d in the Z-direction. As represented in FIG. 1J, the inner surface 124 of cover 120 may be aligned with the back plate 132 of the device case 110 by placing the projection 123 in alignment with and into a recess 134 in the back plate 134. Of course, it should be understood that such projection and recess is exemplary only, and not intended to be limiting. The placement of the projection and recess may be swapped, e.g., the cover 120 may include a recess and the device case 110 may include a projection for insertion into the recess. Alternatively, other structural alignment features or complimentary portions may also be used for aligning and/or removably attaching the interchangeable accessory cover 120 and device case 110 together. For example, in one embodiment, a magnetic attachment may be used by providing magnets on the inner surface 124 and back plate 132, or by forming structural features such as projection 123 and recess 134 out of magnetic materials.

To further secure the interchangeable accessory cover 120 to the device case 110, additional mechanical mechanisms or devices may be used. In such cases, the projection 123 and recess 134 may be used as alignment features. In the illustrated embodiment of FIGS. 1G-1J, the interchangeable accessory cover 120 also includes one or more holes 128 and the back plate 132 includes one or more receiving holes 138. In an embodiment, the receiving holes 138 are threaded. The holes 128 of the accessory cover 120 may be aligned with the receiving holes 138 on the back plate 132 of the device case 110 and configured to receive screws 122 (see FIG. 1G) therethrough that are screwed into and secured onto threads of the receiving holes 138, thereby securing the interchangeable accessory cover 120 to the device case 110.

The holes 128 and 138 may be provided as an alternative to or without the projection 123 and recess 134 shown in the cover 120 and device case 110, in accordance with an embodiment.

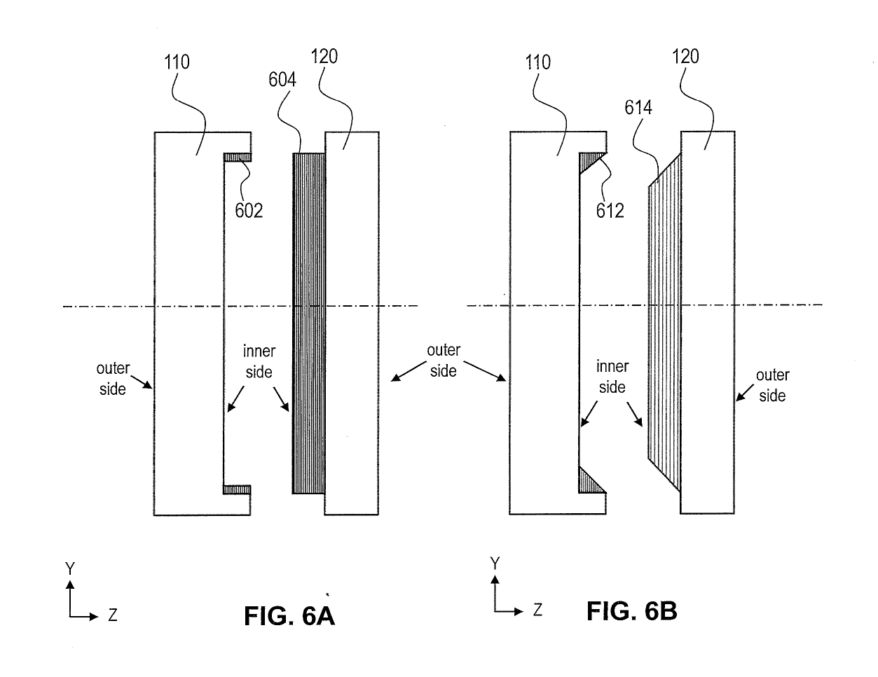

Of course, the use of aligned holes and screws as the removable attachment mechanism for interchangeable accessory cover 120 is not intended to be limiting. FIGS. 6A-10B show schematic representations of alternate designs and (mechanical) mechanisms for attaching device case 110 and an interchangeable accessory cover 120 together to form the wearable device 100. For example, FIGS. 6A and 6B illustrate embodiments wherein threaded attachment mechanisms are used in the wearable device. In one embodiment, shown in FIG. 6A, the device case 110 may include a recess 602 with threads formed along the perimeter of a recess on an inner side of the device case 110. The cover 120 may include a projection 604 projecting from an inner side that also has threads. The threads of the recess 602 and the shaft 604 are compatible with each other and form a secure attachment when screwed together. In another embodiment, as shown in FIG. 6B, the device case 110 may include a tapered recess 612 with threads formed along the perimeter of the tapered recess 612 on an inner side of the device case 110. The cover 120 may include a tapered projection 614 projecting from an inner side with external threads thereon. The shape and dimensions of the recess and the shaft along with the threads are such that the device case 110 and the cover 120 may be aligned so that the threads on the device case and the cover engage with each other and form a secure attachment when screwed together.

In another embodiment, the device case 110 may include external threads (not shown) formed along, for example, an outside perimeter or edge 106, while the cover 120 may include internal threads for connection therewith.

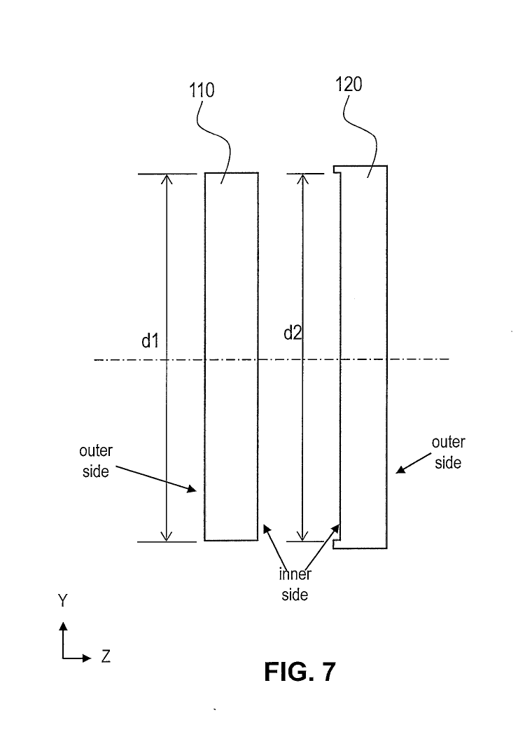

FIG. 7 illustrates another attachment mechanism for parts of the wearable device according to another embodiment. The cover 120 may include an extension along an edge on its inner side that forms a recess for receiving a portion of the device case 110 therein. In one embodiment, a dimension d2 (e.g., an inner diameter) measured within the edge is less than or substantially equal to a dimension d1 associated with the device case 110 (e.g. outer diameter of the device case 110). Then, the edge of the cover 120 may be placed around the perimeter of the device case 110, for receipt of the device within the formed recess of the cover. In one embodiment, the edge includes a snap fit attachment mechanism configured to be snapped into place over at least a portion of the edge 106 of the device case 110.

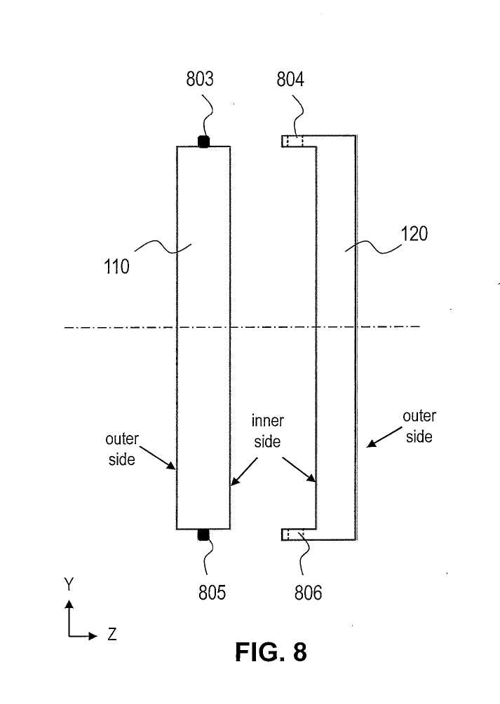

FIG. 8 illustrates another embodiment of an attachment mechanism for parts of the wearable device that includes at least one retractable pin and a corresponding receiving area. In the exemplary illustration, the device case 110 may include pins 803 and 805 projecting outward (e.g., along the Y-direction) from the perimeter or edge 106 of the device case 110. One or more of the pins 803 and 805 may be spring loaded, thus allowing the pins to retract along the length (e.g., in Y-direction) upon application of force and extend when the force is released. The cover 120 may include projections extending from an inner side of the cover, the projections including holes 804 and 806 therein which are configured to receive the pins 803 and 805 of the device case 110. The cover 120 and the device case 110 may be aligned and the pins 803 and 805 may be compressed such the pin 803 may be aligned and inserted into hole 804 and pin 805 is aligned and inserted into hole 806.

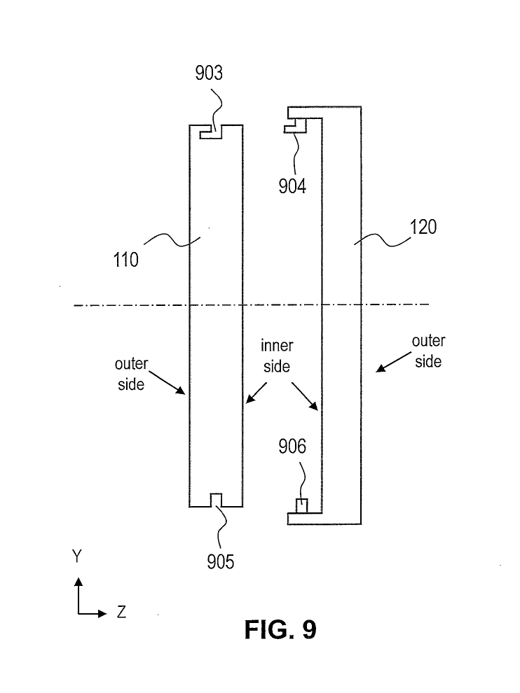

FIG. 9 illustrates a locking attachment mechanism including one or more lock pins 904 and 906 and corresponding receiving slots 903 and 905, respectively, for said lock pins 904 and 906. The cover 120 may include projections extending from an inner side of the cover, for example, and pins 904 and 906 may extend from the projections towards a center of the cover 120. The lock pins may be of different shape and size (as shown) or of similar shape and size, and are not intended to be limiting. The slots 903 and 905 having a shape and size corresponding to the lock pins 904 and 906. The slots 903 and 905 may be formed along the perimeter of the device case 110, for example, e.g., extending into the edge 106 and housing. The cover 120 and the device case 110 may be aligned and the lock pins 904 and 906 are aligned with and inserted into slots 903 and 905.

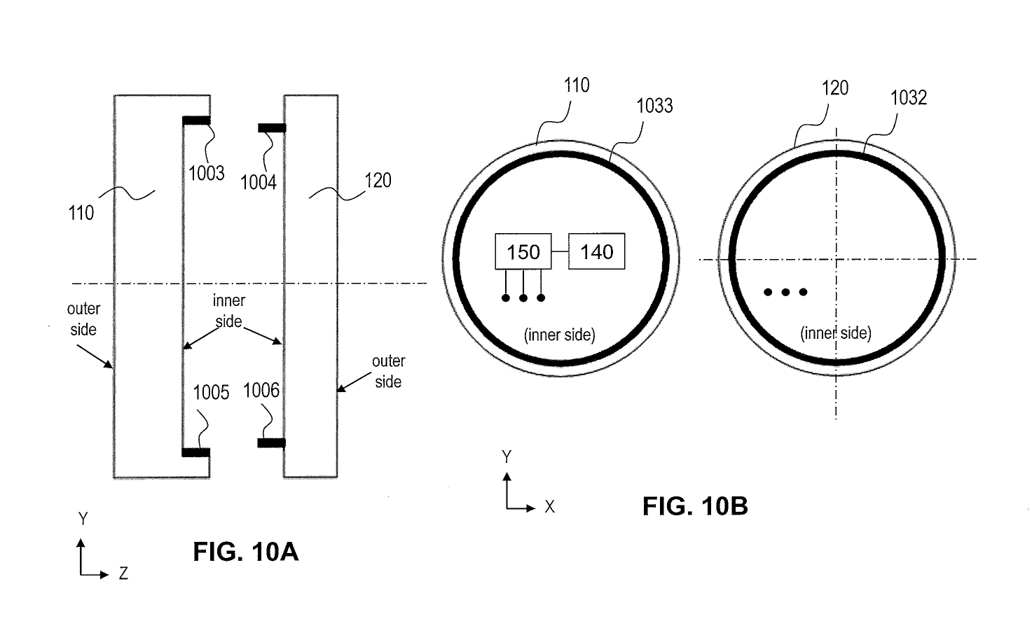

FIGS. 10A and 10B illustrates examples of a magnetic attachment for the wearable device in accordance with embodiments herein. FIG. 10A illustrates one embodiment of the device case 110 and cover 120 having magnets. In an embodiment, the device case 110 may be lined with magnets 1003 and 1005 or a magnetic material on an inner side of the device case 110. For example, the device case 110 may include a recess with the magnets 1003 and 1005 disposed within the recess. In an embodiment, the magnet 1003 and 1005 may be a rectangular piece or a circular piece that may fit within the recess. The cover 120 may also include magnets (or magnetic material) 1004 and 1006 projecting perpendicularly from an inner surface. Furthermore, the magnets 1004 and 1006 may be located relatively below the magnets 1003 and 1005 of the device case 110 when the device case 110 and cover 120 are aligned along a center axis. When assembled, the magnet 1004 of the cover 120 is magnetically coupled the magnet 1003 of the device case 110 and the magnet 1006 of the cover 120 is magnetically coupled to the magnet 1005 of the device case 110, thus creating a magnetic coupling to securely attach the cover 120 to the device case 110.

FIG. 10B illustrates an inner side of the device case 110 and the cover 120 lined with magnets, according to another embodiment. The device case 110 may include a magnet (or a magnetic material) 1033 lined on an inner surface in the proximity of the perimeter of the device case 110. In an embodiment, the magnet 1033 may be located within a recess formed on the inner surface of the device case 110. Similarly, the cover 120 may include a magnet (or magnetic material) 1032 lined on an inner surface in the proximity of the perimeter of cover 120. Additionally or alternatively, the magnet 1032 may be located within a recess formed on the inner surface of the cover 120. Furthermore, the magnet 1032 of the cover 120 is located such that when assembled with the device case 110, the magnet 1032 and 1033 are partially or completely in contact with each other thus creating a magnetic coupling to securely attach the cover 120 to the device case 110.

It should be understood that the schematic illustrations of FIGS. 6A-10B are for illustrative purposes only and not intended to be limiting. It should also be understood to one of ordinary skill in the art that the projections and recesses may be altered between the device case 110 and the cover 120, e.g., instead of cover including projections, the device case includes projections. In addition, other mechanisms for attaching. Although a variety of complimentary designs are shown in FIGS. 6A-10B, the attachment of device case 110 and cover 120 is not limited to such a configuration.

Referring back to the illustrated embodiment of FIG. 1G, for example, in one embodiment, the interchangeable accessory cover 120 has its own electrical connector portion or electrical contact portion 125 that has one or more electrical contact points. Throughout this disclosure, with regards to each accessory cover described herein, the electrical connector portion is referred to as an “electrical contact portion.” However, this is for explanatory purposes only. It should be understood that such an electrical contact portion may be similar to if not the same as the electrical connector portion of the device case in that it functions to establish electrical communication with the processor, sensors, communication circuitry, etc. as well as to establish communication with an external device (e.g., a charging station, a base, a server). In an embodiment, the electrical connectors of electrical connector portion 125 may be made of electrically conducting material (e.g., copper, aluminum, or other metal). The electrical connectors may take several forms and shapes, for example, the electrical connector may be in the form of wires, and/or a strip, may be flexible and/or may be etched on a surface of the interchangeable accessory cover 120. In an embodiment, the electrical connectors may be contact points or a contact surface. In an embodiment, the electrical connectors may include wire with terminals having flat end connected by a wire or a strip. In another embodiment, the terminals may be connected to contact pins, for example, by clamping crimping, soldering, screw termination, etc. In yet another embodiment, the electrical connectors may be ring-shaped or a hollow cylinder such that an electrical contact may be established via a pin or wire type of electrical connector portion of the device case. Also, generally, throughout this disclosure, each electrical contact portion (e.g., 125, 225, etc.) is depicted and described as being of similar shape and configuration to that of the electrical connector portion (155). However, it should be understood that, not only may the shape, configuration, number of wires, etc. change for both types of portions, but the electrical contact portion(s) may have a different shape, configuration, number of wires, etc. as compared to the electrical connector portion (155). Despite its configuration, as explained herein, the electrical contact portion associated with each type of accessory cover may be used to establish a connection with the processor 150, which may be via electrical connector portion 155, for example.

The electrical contact portion 125 may be provided in an area 135 on the accessory cover 120 that may be configured for alignment relative to a location of the electrical connector portion 155, in accordance with an embodiment. In one embodiment, the electrical contact points of the electrical contact portion 125 extend between the front portion 124 and the back portion 126, such that the electrical contact points are accessible via the back portion 126 when the interchangeable accessory cover 120 is attached to the device case 110. The electrical contact portion 125 may be formed from wires that are inserted through holes of the plate of cover 120, wherein an end of each wire is accessible on either side of the plate. In the exemplary illustrated embodiments of the figures, the electrical contact portion 125 includes four electrical wire contact points. In a similar manner as described previously with respect to the connector portion 155, each of the wires of the contact portion 125 may have separate functions, e.g., charging, data transfer, and designed to operate in a similar manner. As such, the electrical contact portion 125 may be used to charge the wearable device 100 as well as transfer data or health related information collected by the wearable device 100 to the external device.

More specifically, the electrical contact points of the electrical contact portion 125 of the interchangeable accessory cover 120 may be configured for alignment with the wires of the electrical connector portion 155 on the device case 110 such that they are in electrical communication. As shown in the Figures, the electrical contact points—shown as four circles in FIG. 1H—on the front portion 124 of the interchangeable accessory cover 120 may be configured for alignment with and/or connection with the electrical connector 155 on the back plate 132 of the device case 110, when the accessory cover 120 is secured thereon. In one embodiment, part of the electrical contact portion 125 may be placed in direct contact with the electrical connector portion 155. Accordingly, an electrical connection between the processor 150 and the external device may be established using the electrical contact points that are accessible (on the back portion 126) via the electrical contact portion 125 of the interchangeable accessory cover 120. Measurements collected by the measurement device(s) 140 or sensor(s) and processed by the processor 150 may be communicated to the external device via the interchangeable accessory cover 120. In addition, data or information may be communicated to the processor 150 and/or electrical components of the wearable device 100 from the external device via using the electrical contact portion 125 of the accessory cover 120 (e.g., aligning the contact portion 125 on an electrical connector of the external device).

Although the electrical contact portion 125 and connector portion 155 are generally described as being parts containing wires that are aligned, it should be understood that such a configuration is not intended to be limiting. For example, in one embodiment, the electrical contact portion 125 on the accessory cover 120 may project from its inner surface 124 for insertion into a receiving area or hole within the device case 110. The electrical contact portion 125 may be passed through the hole to connect to electrical connectors 155 contained within the device case 110. In another embodiment, the device case 110 may include a plurality of holes, one hole for receiving each part of the electrical connector portion 125. In yet another embodiment, the electrical connector portion 155 itself or its wires may project away from a back surface of the device case 110. In one embodiment, the connector portion 155 and/or its wires may be inserted into a receiving area or holes of the accessory cover 120 such that the portion 155 and/or its wires are accessible on the back portion 126 of the cover 120.

In still yet another embodiment, instead of providing electrical contact portion 125, the accessory cover 120 may include an opening, a hole, a cutout portion, or a shape (e.g., either in its edge or the shape of the body itself) for allowing access to the electrical connector portion 155 of the device case 110 when the cover 120 is attached thereto. Such an opening, hole, portion, etc. may be similarly shaped to the electrical connector portion 155, for example. Alternatively, the opening, hole, etc. of the accessory cover 120 may be larger than the area of the electrical connector portion 155.

The materials and manufacturing methods used to form the housing of the device case 110 and interchangeable accessory cover 120 are not limited. The housing of the device case 110 and the accessory cover 120 may be made of similar material, for example, aluminum, steel, gold, silver, other metallic material, plastic, and formed using a similar manufacturing process, e.g., injection molding or casting. In an embodiment, the accessory cover 120 may be made of a different material than the housing of the device case 110. For example, the accessory cover 120 may be made of plastic while the housing is made of metal. Also, the housing of the device case 110 and/or accessory cover 120 may be formed from multiple materials. In some instances, the materials or parts of the device case 110 and/or cover 120 may be opaque or transparent. Using a transparent portion may provide visibility to components, such as the circuitry or sensors within the device case, for example.

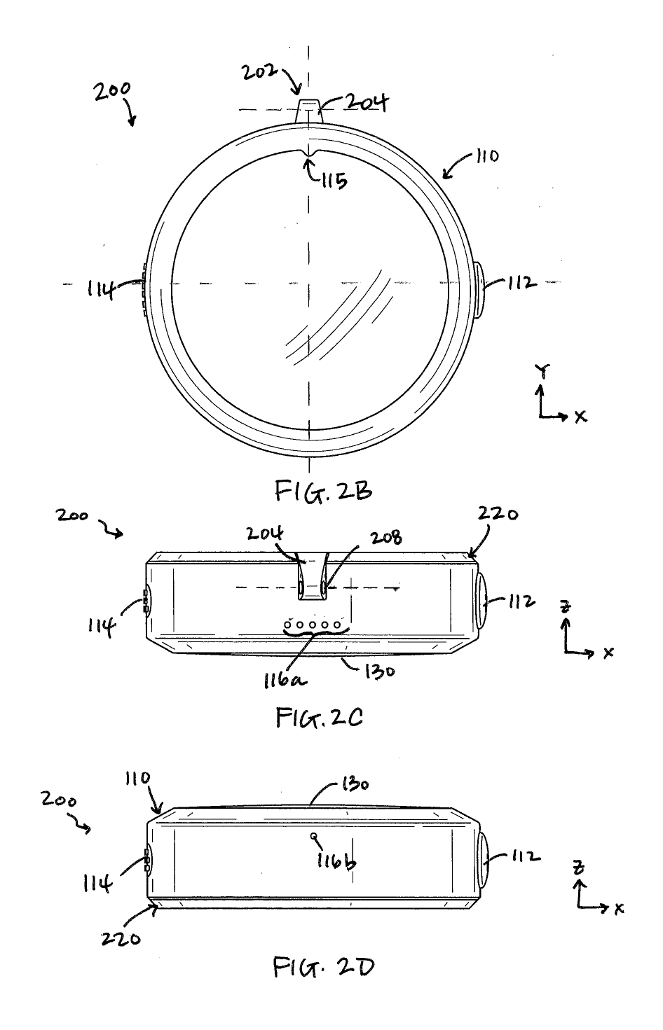

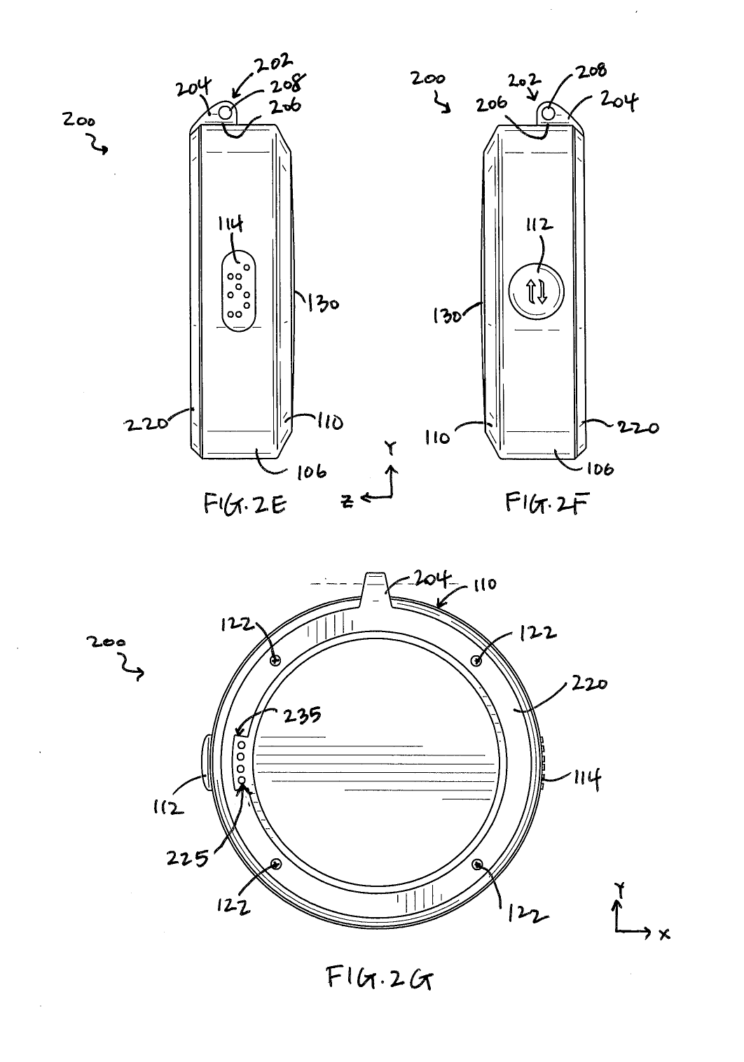

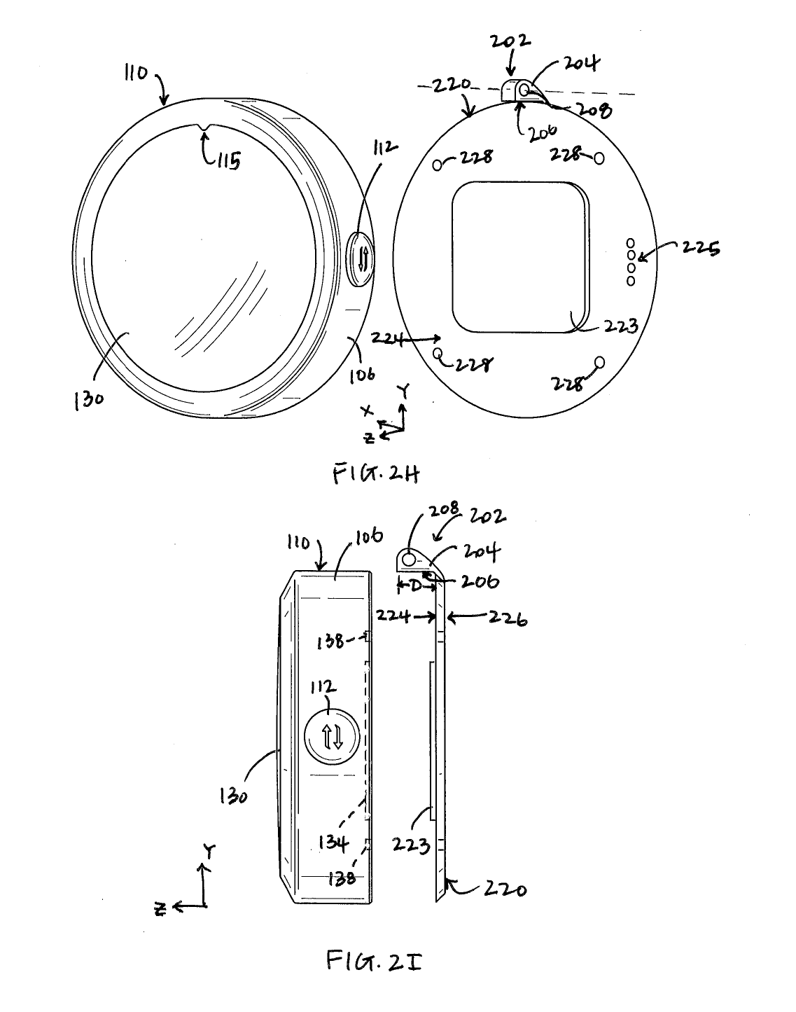

Moreover, the wearable device 100 as disclosed herein is designed to be interchangeable in that different accessories may be provided as part of the wearable device. In accordance with an embodiment, the interchangeable accessory cover 120 may be a first type of cover, which may be removed from the device case 110 and replaced with one or more other types of accessory covers, as discussed with respect to the remaining figures. That is, the device case 110 remains the same while the accessory cover is changed. As shown and described below with reference to different embodiments in FIGS. 2A-5G, accessory covers may be removably attached to a back portion (e.g., using back plate 132) of the device case 110. Changing the back portion is just one option, however. As previously noted, an edge or sides of the device case 110 may be used for attachment purposes. Additionally, the illustrated embodiments and accessories are intended to be exemplary and are not intended to limit the type or use of only those accessories illustrated. It should be understood that other types of interchangeable accessory covers that include, e.g., a keychain, a keyring attachment, a ring, a strap for wrist or shoulder, etc. may be used.

For purposes of clarity and brevity, like elements and components throughout the Figures are labeled with same designations and numbering as discussed with reference to FIGS. 1A-1K. Thus, although not discussed entirely in detail herein, one of ordinary skill in the art should understand that various features associated with the wearable device 100 as shown in FIGS. 2A-10B may be similar to those features previously discussed. Additionally, it should be understood that the features shown in each of the individual figures is not meant to be limited solely to the illustrated embodiments. That is, the features described throughout this disclosure may be interchanged and/or used with other embodiments than those they are shown and/or described with reference to.

FIG. 2A is an exploded, back side angled view of a second type of interchangeable accessory cover 220 and the device case 110 that may be attached together to form a wearable device 200, in accordance with another embodiment of this disclosure. FIGS. 2B-2G illustrate a front, a top, a bottom, a first (left) side, a second (right) side, and a back, respectively, of the wearable device 200. Interchangeable accessory cover 220 includes a necklace attachment portion 202 that allows a user to wear the wearable device 200 as a necklace or pendant around their neck, for example. In some cases, wearable device 200 may be worn around a user's wrist. In a similar manner to interchangeable accessory cover 120, this second interchangeable accessory cover 220 is configured to provide accessibility to the electrical connector portion 155 on the back of the device case 110, when the interchangeable accessory cover 220 is attached thereto. In one embodiment, the device case 110 and the accessory cover 220 may be communicably coupled to each other such that the wearable device 200 may establish an electrical connection as well as communication link with an external device when the cover 220 is placed into electrical contact with the external device.

The second interchangeable accessory cover 220 may have a portion provided in the form of a plate for placement on the back of the device case. The plate of the accessory cover 220 has an inner surface 224 (see FIG. 2H) and an outer surface 226 (see FIG. 2A), and the necklace attachment portion 202 extending from the plate. Like accessory cover 120, the plate of accessory cover 220 may include a beveled edge that is provided at an angle 3. In an embodiment, the plate itself, its inner surface 224, and/or outer surface 226 may be similarly shaped to the housing of device case 110—or at least its back plate 132—for example, both have a substantially circular shape or any other shape (e.g., square, rectangular, oval, etc.) that correspond to each other. In one embodiment, the plate associated with cover 220 is substantially disc shaped.

In addition, shown in FIG. 2H, the interchangeable accessory cover 220 may include a projection 223 (see FIG. 2H) that is similar to projection 123, for alignment with a complimentary sized recess 134 (shown in FIG. 2A) on a back portion of the device case 110, in accordance with an embodiment. Furthermore, the second interchangeable accessory cover 220 may also include one or more holes 228 that may be aligned with the receiving holes 138 on the back plate 132 of the device case 110 in a similar manner as described with respect to holes 128 of accessory cover 120. The holes 228 may be configured to receive screws 122 (see FIG. 2G) therethrough that are screwed into and secured onto threads of the receiving holes 138, thereby securing the interchangeable accessory cover 320 to the device case 110. The holes 228 may be provided as an alternative to or without the projection 223 shown in the cover 220, in accordance with an embodiment. Again, the use of holes and screws as the removable attachment mechanism for interchangeable accessory cover 220 is not intended to be limiting. As described in detail previously with regards to FIGS. 6A through 10B, it should be appreciated that the third interchangeable accessory cover 220 is not limited to screw-type attachment and may be modified to use other means of removable attachments such as those described with reference thereto.

Hence, either the first or second interchangeable accessory covers 120 or 220 may be removed and replaced by the third interchangeable accessory cover 320, or vice-versa. Accordingly, the accessory associated with the device case 110 may be changed for the wearable device so that it may be worn on different parts of a user's body.

An electrical connector portion or electrical contact portion 225, which is equivalent to the previously described electrical contact portion 125 and its embodiments, is disposed on the outer surface 226 of the accessory cover 220. As shown in FIGS. 2A, 2G, and 2H, for example, in one embodiment, the interchangeable accessory cover 220 has its own an electrical contact portion 225 that has one or more electrical contact points. The electrical contact portion 225 may be provided in an area 235 on the accessory cover 220 that may be configured for alignment relative to a location of the electrical connector portion 155, in accordance with an embodiment. In one embodiment, the electrical contact points extend between the front portion or inner surface 224 and the back portion or outer surface 226, such that the electrical contact points are accessible via the outer surface 226/back portion when the interchangeable accessory cover 220 is attached to the device case 110. In another embodiment, holes are provided for receipt of wires therein. Since the features relating to electrical contact portion 225 are similar to the electrical contact portion 125 (e.g., use of wires, four electrical wire contact points, functions (charging, data transfer), etc.) which are described in detail above with respect to FIGS. 1A-1J, they are not all necessarily repeated here. Nonetheless, it should be understood that the accessory cover 220 may include any number of features described with reference to electrical contact portion 125.

The electrical contact points of the electrical contact portion 225 of the accessory cover 220 may be configured for alignment with the wires of the electrical connector portion 155 on the device case 110 such that they are in electrical communication. In one embodiment, part of the electrical contact portion 225 may be placed in direct contact with the electrical connector portion 155. Accordingly, an electrical connection between the processor 150 and the external device may be established using the electrical contact points of electrical contact portion 225. Measurements collected by the measurement device(s) 140 or sensor(s) and processed by the processor 150 may be communicated to the external device via the interchangeable accessory cover 220. In addition, data or information may be communicated to the processor 150 and/or electrical components of the wearable device 200 from the external device via using the electrical contact portion 225 of the accessory cover 220.

Of course, it should also be noted that in yet another embodiment, instead of providing electrical contact portion 225, the accessory cover 220 may include an opening, a hole, a cutout portion, or a shape for allowing access to the electrical connector portion 155 of the device case 110 when the cover 220 is attached thereto, as previously described.

The materials and manufacturing methods used to form the interchangeable accessory cover 220 are not limited. The accessory cover 220 may be made of similar material as the device case 110, for example, aluminum, steel, gold, silver, other metallic material, plastic, and formed using a similar manufacturing process, e.g., injection molding or casting. In an embodiment, the accessory cover 220 may be made of a different material than the housing of the device case 110. Also, the accessory cover 220 may be formed from multiple materials.

As shown in FIG. 2I, for example, the necklace attachment portion 202 of accessory cover 220 extends outwardly from an edge thereof. The necklace attachment portion 202 may include a lug 204 that is connected or attached to a beveled edge of the plate, in accordance with an embodiment. In the illustrated embodiment, the lug 204 extends forwardly at a distance D (see FIG. 2I) from the inner surface 224 of the plate in the Z-direction (away from the outer surface 226), which is visible when viewed from the X-direction. In one embodiment, the lug 204 has a flat surface 206 that is configured for placement against the edge 106 of the device case 110 when the accessory cover 220 is attached thereto (see FIGS. 2E and 2F), so the lug 204 is substantially flush with respect to the perimeter or circumference of the edge 106 of the device case 110.

The lug 204 has a receiving hole 208 configured to receive a chain, a string, a wire, or other type of cord or band (which may or may not be flexible), generally represented as cord 210, therethrough. As previously noted, the cord 210 allows a user to wear the wearable device 200 like a necklace around a neck, around a wrist, or other body part. The receiving hole 208 may be positioned forwardly relative to the inner surface 224, as shown in FIG. 2I, such that when assembled, the receiving hole 208 is positioned near or closer to a center or halfway point of a width of the edge 106.

When the wearable device 200 is assembled, the lug 204 may be positioned in a location that aligns with an orientation indicator 115 in the 12 o'clock position of the device case 110, e.g., along a vertical centerline of the device 200 (see FIG. 2B) in the Y-direction, when the cover 220 is attached to the device case 110. An axis through the receiving hole 208 runs parallel to a horizontal centerline in the X-direction (e.g., see FIGS. 2B and 2C).

The lug 204 may be formed as an integral part of the second interchangeable accessory cover 220 or as a separate attachment that is secured to the plate of the cover. In an embodiment, the lug 204 may be removably attached to the plate of the second interchangeable accessory cover 220, for example, via a snap fit connection, screw-type connectors, or other mechanical attachment. In an embodiment, the lug 204 may be fixed to the interchangeable accessory cover 220. Furthermore, in other embodiments, the lug 204 may be hingedly attached or rotatably attached to the interchangeable accessory cover 220, allowing the lug 204 to rotate relative to the interchangeable accessory cover 220 and/or the device case 210.

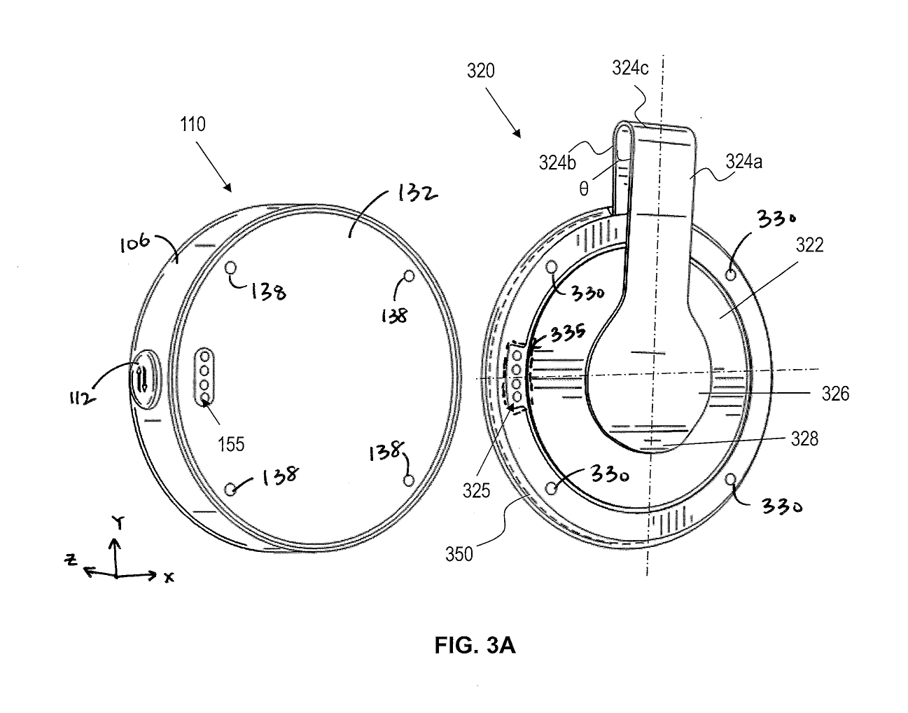

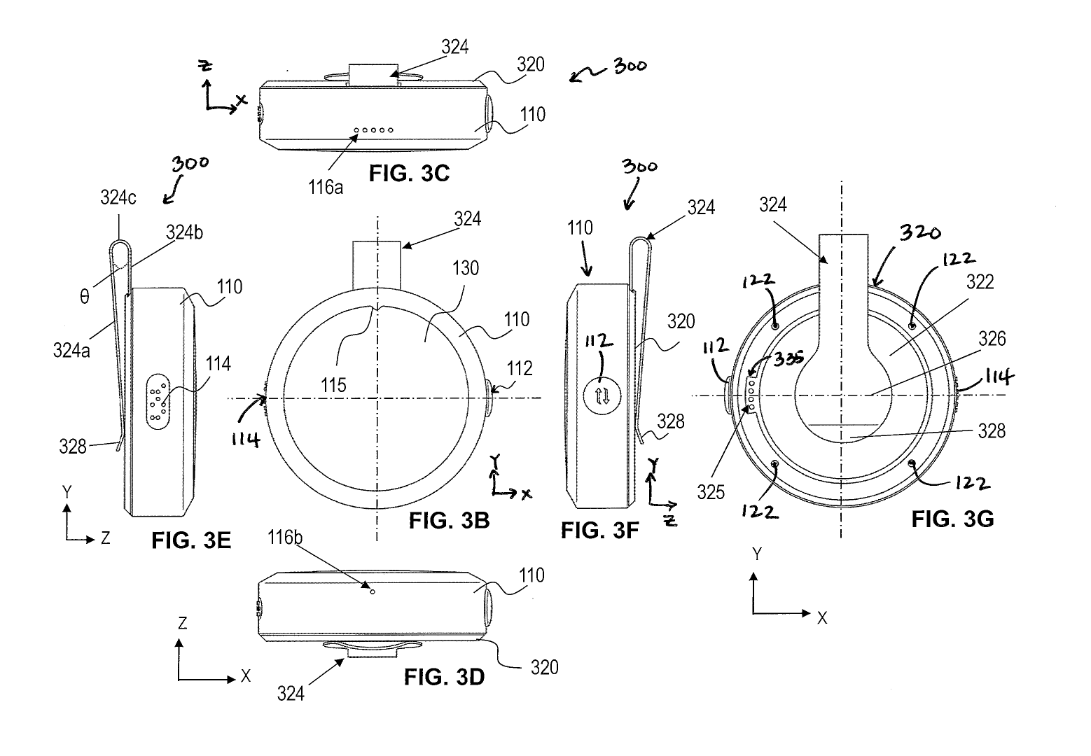

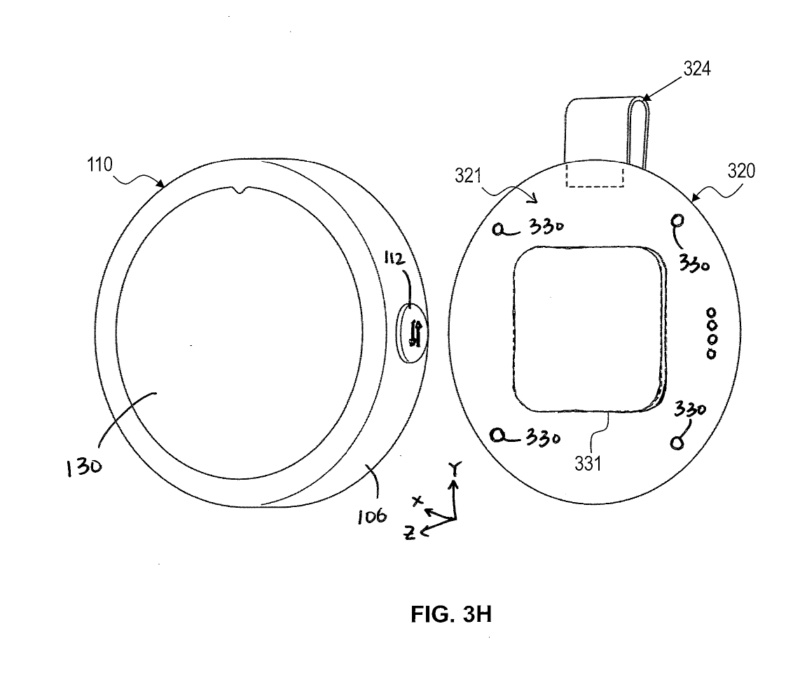

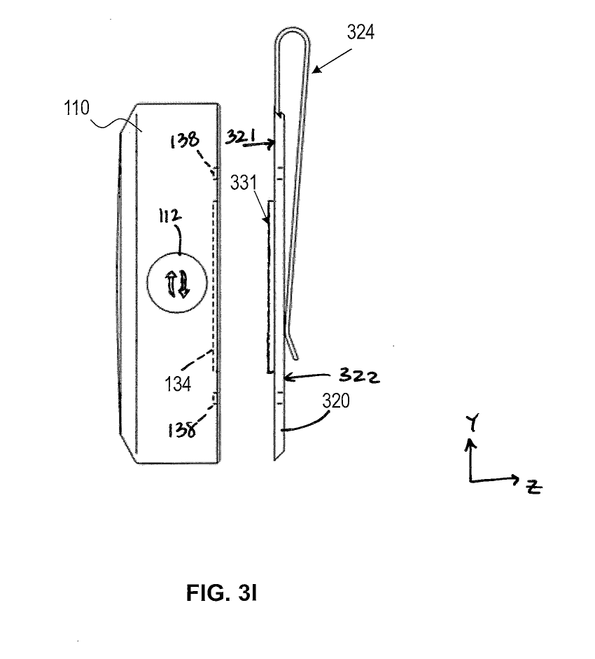

A third type of interchangeable accessory cover 320 along with device case 110 is shown in FIGS. 3A-3I, forming a wearable device 300 according to another embodiment of the disclosure. Much like interchangeable accessory cover 120, interchangeable accessory cover 320 is configured to provide accessibility to the electrical connector portion 155 on the back of the device case 110, when the interchangeable accessory cover 320 is attached thereto. In one embodiment, the device case 110 and the accessory cover 320 may be communicably coupled to each other such that the wearable device 300 may establish an electrical connection as well as communication link with an external device when the cover 320 is placed into electrical contact with the external device. The third interchangeable accessory cover 320 may have a portion provided in the form of a plate for placement on the back of the device case. The plate of the accessory cover 320 has an inner surface 321 (see FIG. 3H) and an outer surface 322 (see FIG. 3A), and a clip 324 connected or attached thereto. Like accessory cover 120, the plate of the accessory cover 320 may include a beveled edge that is provided at an angle 3. In an embodiment, the plate itself, its inner surface 321, and/or outer surface 322 may be similarly shaped to the housing of device case 110—or at least its back plate 132—for example, both have a substantially circular shape or any other shape (e.g., square, rectangular, oval, etc.) that correspond to each other. In one embodiment, the plate associated with cover 320 is substantially disc shaped.

In addition, the interchangeable accessory cover 320 may include a projection 331 (see FIG. 3H) that is similar to projection 123, for alignment with a recess (not shown in FIG. 3A) on a back portion of the device case 110, in accordance with an embodiment. It should be understood that the device case 110 may include a recess similar to recess 134 that is similar in shape and size as the projection 331. Furthermore, the third interchangeable accessory cover 320 may also include one or more holes 321 that may be aligned with the receiving holes 138 on the back plate 132 of the device case 110 in a similar manner as described with respect to holes 128 of accessory cover 120. The holes 321 may be configured to receive screws 122 (see FIG. 3G) therethrough that are screwed into and secured onto threads of the receiving holes 138, thereby securing the interchangeable accessory cover 320 to the device case 110. The holes 321 may be provided as an alternative to or without the projection 331 shown in the cover 320, in accordance with an embodiment. Of course, the use of holes and screws as the removable attachment mechanism for interchangeable accessory cover 320 is not intended to be limiting. As described in detail previously with regards to FIGS. 6A through 10B, it should be appreciated that the third interchangeable accessory cover 320 is not limited to screw-type attachment and may be modified to use other means of removable attachments such as those described with reference thereto.

As such, it should be understood that the second interchangeable accessory cover 220 may not only be used with the device case 110, but it may replace the (first) interchangeable accessory cover 120, or vice-versa. Each of the covers 120 and 220 may be removed and replaced with the other. Accordingly, the accessory associated with the device case 110 may be changed for the wearable device so that it may be worn on different parts of a user's body.

An electrical connector portion or electrical contact portion 325, which is equivalent to the previously described electrical contact portion 125 and its embodiments, is disposed on the outer surface 322 of the accessory cover 320. As shown in FIGS. 3A, 3G, and 3H, for example, in one embodiment, the interchangeable accessory cover 320 has its own an electrical contact portion 325 that has one or more electrical contact points. The electrical contact portion 325 may be provided in an area 335 on the accessory cover 320 that may be configured for alignment relative to a location of the electrical connector portion 155, in accordance with an embodiment. In one embodiment, the electrical contact points extend between the front portion or inner surface 321 and the back portion or outer surface 322, such that the electrical contact points are accessible via the outer surface 322/back portion when the interchangeable accessory cover 320 is attached to the device case 110. In another embodiment, holes are provided for receipt of wires therein. Since the features relating to electrical contact portion 325 are similar to the electrical contact portion 125 (e.g., use of wires, four electrical wire contact points, functions (charging, data transfer), etc.) which are described in detail above with respect to FIGS. 1A-1J, they are not all necessarily repeated here. Nonetheless, it should be understood that the accessory cover 320 may include any number of features described with reference to electrical contact portion 125.

The electrical contact points of the electrical contact portion 325 of the accessory cover 320 may be configured for alignment with the wires of the electrical connector portion 155 on the device case 110 such that they are in electrical communication. In one embodiment, part of the electrical contact portion 325 may be placed in direct contact with the electrical connector portion 155. Accordingly, an electrical connection between the processor 150 and the external device may be established using the electrical contact points of electrical contact portion 325. Measurements collected by the measurement device(s) 140 or sensor(s) and processed by the processor 150 may be communicated to the external device via the interchangeable accessory cover 320. In addition, data or information may be communicated to the processor 150 and/or electrical components of the wearable device 300 from the external device via using the electrical contact portion 325 of the accessory cover 320.

Of course, it should also be noted that in yet another embodiment, instead of providing electrical contact portion 325, the accessory cover 320 may include an opening, a hole, a cutout portion, or a shape for allowing access to the electrical connector portion 155 of the device case 110 when the cover 320 is attached thereto, as previously described.

The materials and manufacturing methods used to form the interchangeable accessory cover 320 are not limited. The accessory cover 320 may be made of similar material as the device case 110, for example, aluminum, steel, gold, silver, other metallic material, plastic, and formed using a similar manufacturing process, e.g., injection molding or casting. In an embodiment, the accessory cover 320 may be made of a different material than the housing of the device case 110. Also, the accessory cover 320 may be formed from multiple materials.

The clip 324 may be a spring-type belt clip or holster clip that includes a resilient spring arm or similar resilient and flexible member, configured to move relative to a back of the plate of the accessory cover 320 to receive and secure a piece of clothing between the arm/member and a back surface of the accessory cover 320. For example, the wearable device 300 may be attached to a belt or a portion of a shirt or pants of a user. As shown in the figures, the clip 324 is provided in its original position (i.e., not in use or grasping clothing).

The clip 324 may have a first arm or leg 324a, a second arm or leg 324b, and a curved portion 324c connecting the two legs 324a and 324b. The first leg 324a is configured to move relatively away from the outer surface 322, and the second leg 324b, to receive a piece of clothing therein, for example.

The second leg 324b may be the portion that attaches the clip 324 to the accessory cover 320. The second leg 324b may be integrally formed with the accessory cover 320, for example, by molding and/or soldering, or fixedly attached, e.g., via adhesive, to a perimeter of the outer surface 322 of the interchangeable accessory cover 320. In one embodiment, the second leg 324b is secured to or in a receiving area provided along the perimeter or beveled edge of the cover 320. As seen in FIG. 3E, the second leg 324b may project or extend outwards (e.g., in a vertical direction or y-direction) from the beveled edge of the accessory cover 320. The second leg 324b may be parallel to a plane across the outer surface 322, for example. The curved portion 324c may be generally C-shaped, U-shaped, or even V-shaped and is spaced from the edge 106 of the device case. The curved portion 324c positions the first leg 324a adjacent to outer surface 322, such that there is an angle θ with respect to the second leg 324b (the angle θ being between the inner facing surfaces of each of the legs). When the first leg 324a is moved relatively away from the outer surface 322 (and thus the second leg 324b), the angle therebetween increases. However, the curved portion 324c is designed to be resilient so that it pulls the first leg 324a back towards the second leg 324b, in some cases back to angle θ. In accordance with an embodiment, the clip 324 may be made from a resilient material such that the curved portion 324c encourages any relative movement of the first leg 324a away from the outer surface 322 and second leg 324b back towards its original position.

Furthermore, in an embodiment, the first leg 324a may include an end portion 326 (see FIGS. 3A and 3G) that has an inner surface configured to rests on the outer surface 322 of the third interchangeable accessory cover 320 in its original position. In an embodiment, the end portion 326 may larger in size and different (or similar) in shape compared to the first leg 324a. For example, the end portion 326 may be substantially circular in shape, while the first and second legs 324a and 324b are linear. Such an end portion 326 provides a larger contact surface for grasping clothing between the outer surface 322 and end portion 326, for example. Of course, the shape of the end portion 326 is exemplary only and not intended to be limiting. The end portion 326 may also include a bent portion 328 that is angled or extends away from the outer surface 322, as seen in FIG. 3F, for example, in one embodiment. The bent portion 328 assists a user in grasping the first legs 324a of the clip 324 such that it may be moved away from the outer surface 322 for receipt of a piece of clothing. In an embodiment, the end portion 326 is positioned such that its center aligns with a center of the device case 110, e.g., a center point of the vertical and horizontal centerlines that extend in the Y- and X-directions. In one embodiment, the bent portion 328 may be positioned below the horizontal centerline (see FIG. 3G).

Accordingly, to secure the wearable device 300 to a user, the end portion 326 is moved away from the outer surface 322, thereby allowing a piece of clothing or belt to slide (e.g., in vertical direction) between the outer surface 322 and the first leg 324a and towards the curved portion 324c. In an embodiment, the resiliency of at least the first leg 324a and curved portion 324c of the clip 324 secures the clothing or belt therein.

Furthermore, in an embodiment, the clip 324 may be configured to be removably attached to the outer surface 322 of the third interchangeable accessory cover 320. For example, the second leg 324b may be snap fitted, screwed or attached by other removable fastening methods. In an embodiment, the third interchangeable accessory cover 320 may include tracks 350 to allow relative motion between the clip 324 and the interchangeable accessory cover 320, such that the clip 324 may be moved along the perimeter of the outer surface 322 of the interchangeable accessory cover 320.

Although the clip 324 of the third interchangeable accessory cover 320 is shown in the Figures such that, in an embodiment, the second leg 324b of the clip 324 is generally aligned with the orientation indicator 115 in the 12 o'clock position when the cover 320 is attached to the device case 110, the placement of the clip 324 along an edge of the plate of the cover 320 is not limited except for allowing access to the electrical contact portion 325. That is, since electrical contact portion 325 is used to establish a connection between the processor 150 of the device case 110 and an external device, it must be accessible. Accordingly, a portion (e.g., 324a) of the clip 324 should not cover or limit access to the electrical contact points of the electrical contact portion 325.

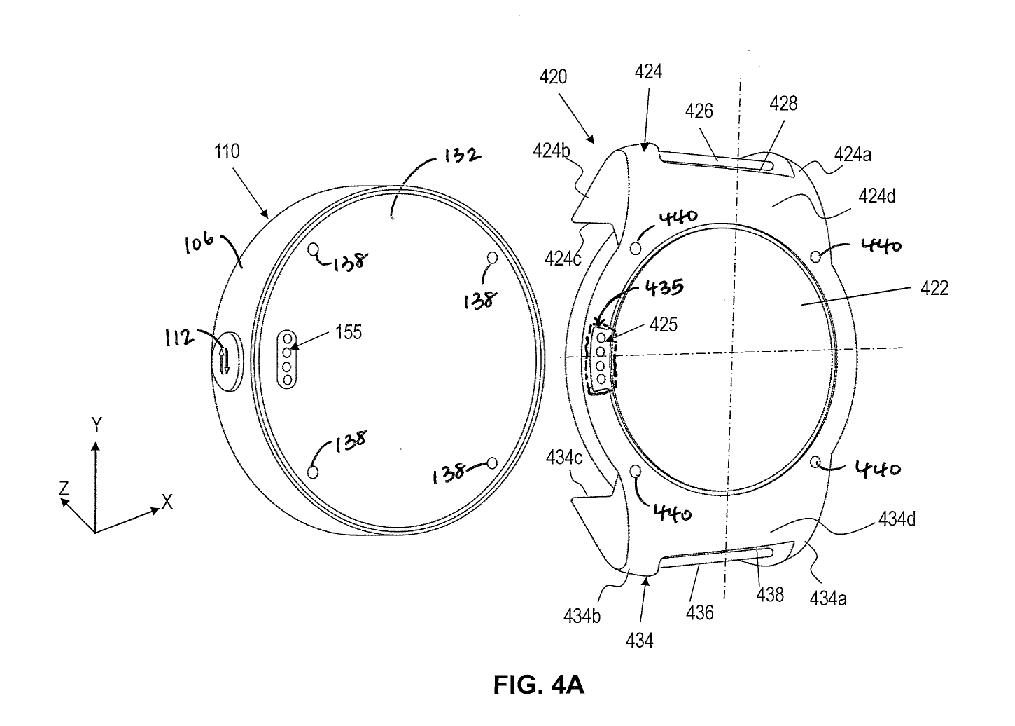

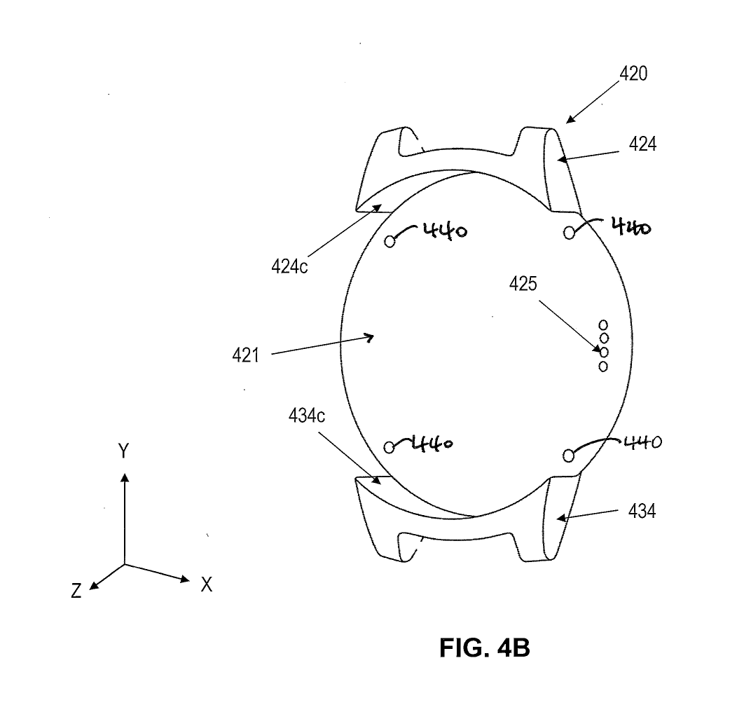

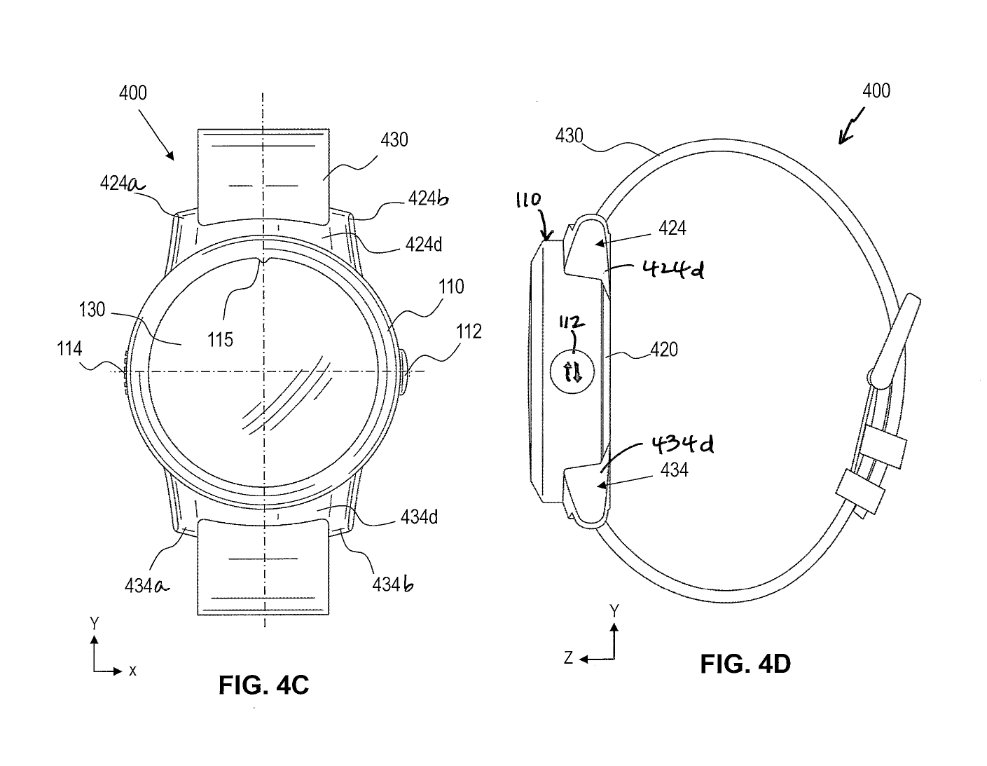

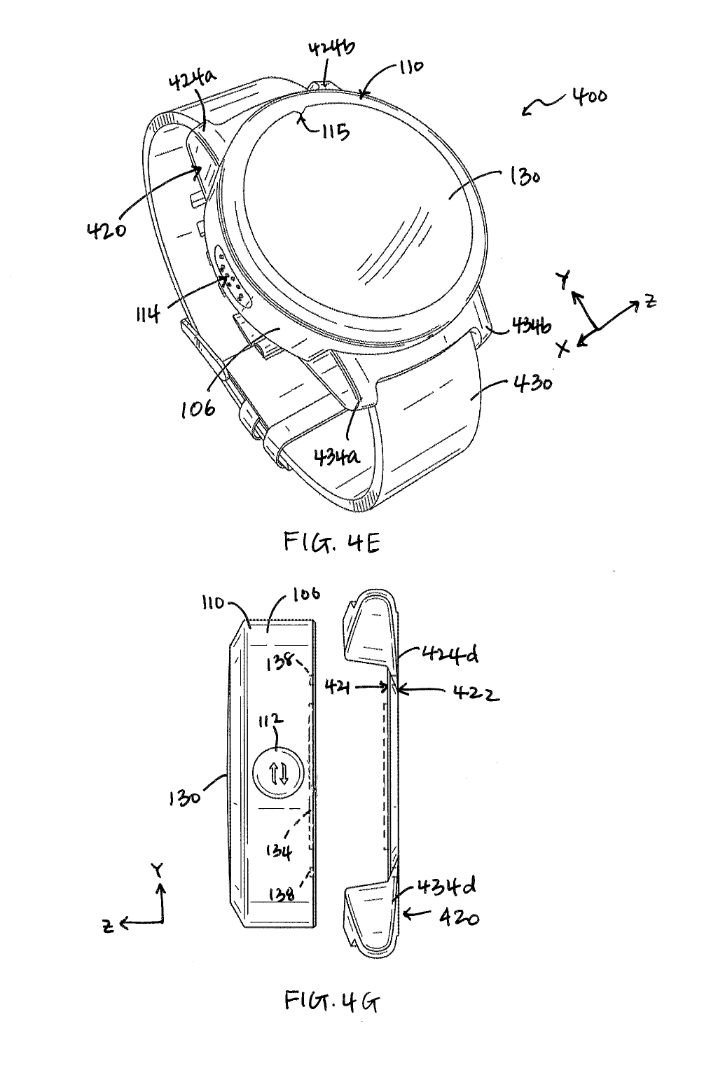

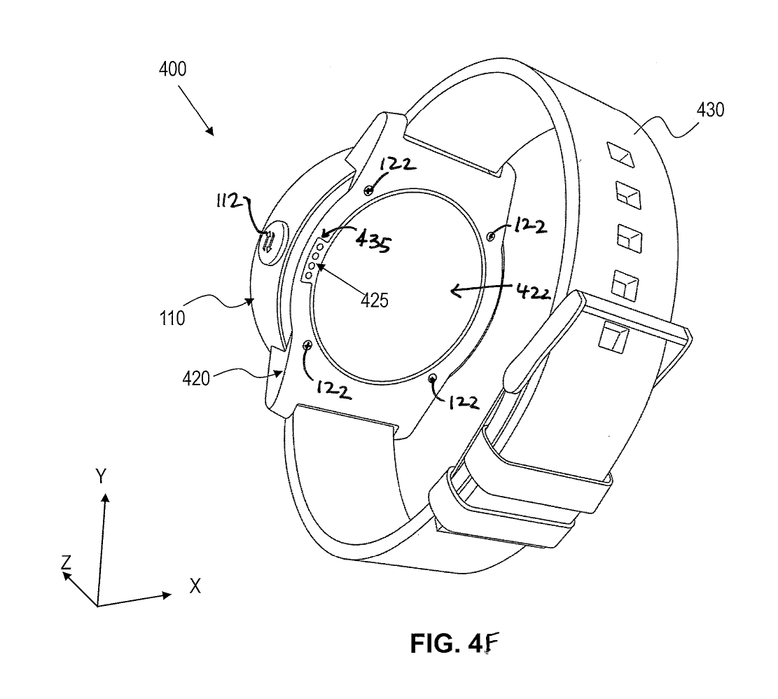

In accordance with another embodiment, a fourth interchangeable accessory cover 420 may be used along with device case 110 as shown in FIGS. 4A-4G, forming a wearable device 400. Much like interchangeable accessory cover 120, interchangeable accessory cover 420 is configured to provide accessibility to the electrical connector portion 155 on the back of the device case 110 when attached thereto. In one embodiment, the device case 110 and the accessory cover 420 may be communicably coupled to each other such that the wearable device 400 may establish an electrical connection as well as communication link with an external device when the cover 400 is placed into electrical contact with the external device. The fourth interchangeable accessory cover 420 may have a portion provided in the form of a plate for placement on the back of the device case. The plate of the accessory cover 420 has an inner surface 421 (see FIG. 4B) and an outer surface 422 (see FIG. 4A). Pairs of lugs that are part of watch lug portions 424 and 434 (described later below) are connected or attached to the plate of the cover 420. In an embodiment, the plate itself, its inner surface 421, and/or outer surface 422 may be similarly shaped to the housing of device case 110—or at least its back plate 132—for example, both have a substantially circular shape or any other shape (e.g., square, rectangular, oval, etc.) that correspond to each other. In one embodiment, the plate associated with cover 420 is substantially disc shaped.

In addition, although not shown in the Figures, it should be understood that the interchangeable accessory cover 420 may include a projection, that is similar to projection 123, for alignment with a recess (also not shown), similar to recess 134, on a back portion of the device case 110, in accordance with an embodiment. Furthermore, the fourth interchangeable accessory cover 420 may also include one or more holes 440 that may be aligned with the receiving holes 138 on the back plate 132 of the device case 110 in a similar manner as described with respect to holes 128 of accessory cover 120. The holes 440 may be configured to receive screws 122 (see FIG. 4F) therethrough that are screwed into and secured onto threads of the receiving holes 138 to secure the interchangeable accessory cover 420 to the device case 110. Of course, the use of holes and screws as the removable attachment mechanism for interchangeable accessory cover 420 is not intended to be limiting, and any other mechanism including those described in detail previously with regards to FIGS. 6A through 10B may be used.

As such, it should be understood that the fourth interchangeable accessory cover 420 may be used with the device case 110, as well as to replace any other interchangeable accessory cover 120, 220, and/or 320, or vice-versa. In this case, the accessory associated with the device case 110 may be changed such that it forms the wearable device 400 for placement around a user's wrist.

An electrical connector portion or electrical contact portion 425, which is equivalent to the previously described electrical contact portion 125 and its embodiments, is disposed on the outer surface 422 of the accessory cover 420. As shown in FIGS. 4A, 4B, and 4F, for example, in one embodiment, the interchangeable accessory cover 420 has its own an electrical contact portion 425 that has one or more electrical contact points. The electrical contact portion 425 may be provided in an area 435 on the accessory cover 420 that may be configured for alignment relative to a location of the electrical connector portion 155, in accordance with an embodiment. In one embodiment, the electrical contact points extend between the front portion or inner surface 421 and the back portion or outer surface 422, such that the electrical contact points are accessible via the outer surface 422/back portion when the interchangeable accessory cover 420 is attached to the device case 110. In another embodiment, holes are provided for receipt of wires therein. Since the features relating to electrical contact portion 425 are similar to the electrical contact portion 125 (e.g., use of wires, four electrical wire contact points, functions (charging, data transfer), etc.) which are described in detail above with respect to FIGS. 1A-1J, they are not all necessarily repeated here. Nonetheless, it should be understood that the accessory cover 420 may include any number of features described with reference to electrical contact portion 125.

The electrical contact points of the electrical contact portion 425 of the accessory cover 420 may be configured for alignment with the wires of the electrical connector portion 155 on the device case 110 such that they are in electrical communication. In one embodiment, part of the electrical contact portion 425 may be placed in direct contact with the electrical connector portion 155. Accordingly, an electrical connection between the processor 150 and the external device may be established using the electrical contact points of electrical contact portion 425. Measurements collected by the measurement device(s) 140 or sensor(s) and processed by the processor 150 may be communicated to the external device via the interchangeable accessory cover 420. In addition, data or information may be communicated to the processor 150 and/or electrical components of the wearable device 400 from the external device via using the electrical contact portion 425 of the accessory cover 420.

Of course, it should also be noted that in yet another embodiment, instead of providing electrical contact portion 425, the accessory cover 420 may include an opening, a hole, a cutout portion, or a shape for allowing access to the electrical connector portion 155 of the device case 110 when the cover 420 is attached thereto, as previously described with reference to cover 120.

Watch lug portions 424 and 434 are of generally similar construction and configured to receive and secure a band 430 (shown in FIGS. 4C-4F) in order to facilitate a user wearing of the wearable device 400 on a wrist, an arm, and/or other body parts. In an embodiment, the lug portions 424 and 434 are located diametrically opposite to each other. Each watch lug portion 424 and 434 has a base portion 424d and 434d, respectively, that extends or projects from the plate of the accessory cover 420, and a pair of lugs 424a, 424b and 434a, 434b, respectively.

In the illustrated embodiment, the base portions 424d and 434d (and thus the pairs of lugs) extend both in the Y-direction (or vertically) and the Z-direction (or axially), when viewed in the X-direction (see FIG. 4G). In an embodiment, the back surface of each base portion 424d and 434d may be aligned with and provided in the same plane of the outer surface 422 (see FIG. 4D), thus extending outward (e.g., in a vertical direction or y-direction) and increasing a surface area of the back portion of the accessory cover 420. In an embodiment, the front surface of each base portion 424d and 434d may be positioned such that it extends from the edge 106 of the device case 110 when assembled thereto.

As shown in FIG. 4B, in accordance with an embodiment, each base portion 424d and 434d may include a curved surface 424c and 434c, respectively, that extends forwardly from the inner surface 421. The curved surfaces 424c and 434c each have an arc or a curvature that cooperates or confirms with the sides or edge 106 of the device case 110. Accordingly, when the accessory cover 420 is attached to the device case 110, the curved surfaces 424c and 434c may be provided along or against the perimeter or edge 106 of the device case 110.

Each lug 422a, 424b and 434a, 434b may extend in a generally perpendicular direction (e.g., in a vertical direction or Y-direction) relative to edge 106, for example, in the wearable device 400. Referring specifically to the base portion 424d, as shown in 4C, for example, the pair of lugs 424a and 424b at the top end of the accessory cover 420 are spaced from one another, e.g., at either side (right and left) of the base portion 424d. A space between the pair of lugs 424a and 424b is configured for receipt of an end of a watch band 430, for example. Specifically, each lug 424a and 424b has a receiving hole therein that is configured to receive an end or a portion of a removable pin 426 therein. The receiving holes are provided in the internal surfaces of the lugs 424a and 424b (i.e., the surfaces that are facing each other), and are horizontally aligned. The receiving holes may be through holes or blind holes in each of the lugs. In another embodiment, the receiving holes may be provided as dimples that are horizontally aligned with each other. To attach a band 430, the pin 426 may be placed through a through hole (not shown) on one end of the band 430, and its ends may be inserted and secured in the receiving holes of the lugs 424a and 424b.

The base portion 434d, also shown in FIG. 4C, includes its pair of lugs 434a and 434b positioned at the bottom end of the accessory cover 420. Similar to base portion 424d, lugs 434a and 434b of base portion 434d are spaced from one another, e.g., at either side (right and left) of the base portion 434d, for receipt of another end of the band therebetween, for example. Lugs 434a and 434b also have receiving holes or dimples provided therein as described above (and thus not repeated here) for placement of a removable pin 436 therein. To attach the other end of the band 430, then, the pin 436 may be placed through a through hole (not shown) on the other/opposite end of the band 430, and its ends may be inserted and secured in the receiving holes of the lugs 434a and 434b.

In an embodiment, gaps 428 and 438 (shown in FIG. 4A) are provided between the pins 426 and 436 and a top surface of the base portions 424d and 434d, respectively, to accommodate the ends of the band.

In one embodiment, the pins 426 and 436 may each be a spring loaded shaft that is configured to compress in some manner, to allow ends of the pins 426 and 436 to be inserted within the receiving holes/dimples before expanding to its original length.

In an embodiment, the lug portions 424 and 434 may be integrally formed with the plate and/or its outer surface 422 (e.g., via molding) or attached thereto (e.g., by molding, soldering, or adhesive) to from an integral structure. However, the materials and manufacturing methods used to form the interchangeable accessory cover 420 are not limited. The accessory cover 420 may be made of similar material as the device case 110, for example, aluminum, steel, gold, silver, other metallic material, plastic, and formed using a similar manufacturing process, e.g., injection molding or casting. In an embodiment, the accessory cover 420 may be made of a different material than the housing of the device case 110. Also, the accessory cover 420 may be formed from multiple materials. Additionally, any type of band 430 may be used with wearable device 400. The band 430 may be formed from leather, plastic, or cloth materials, for example.

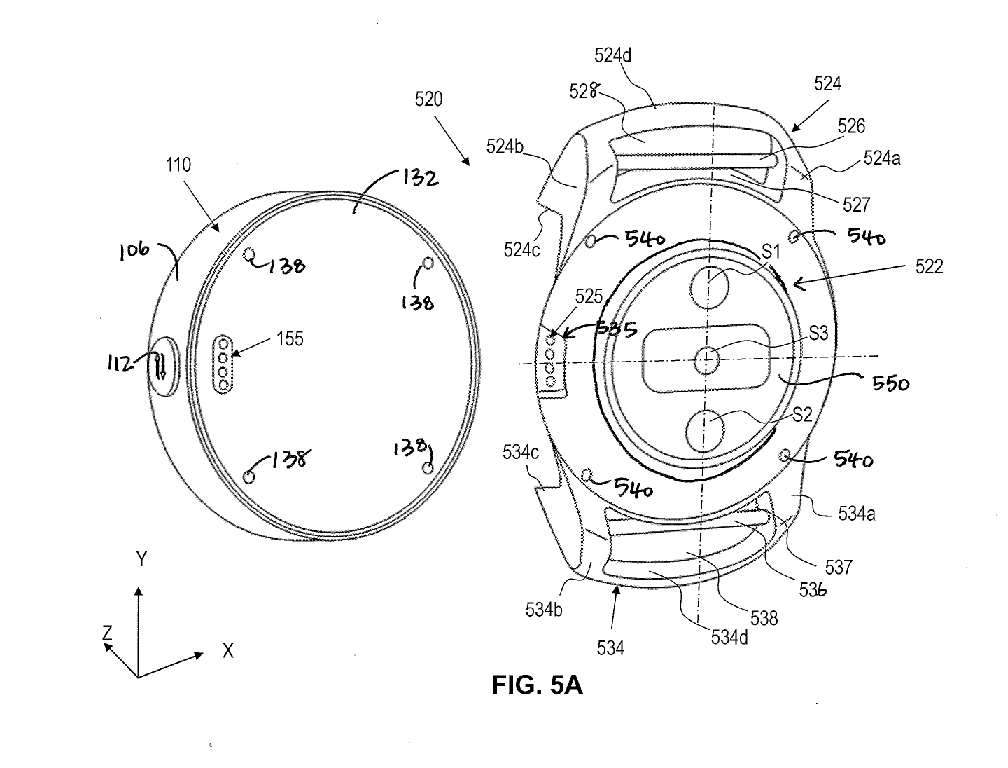

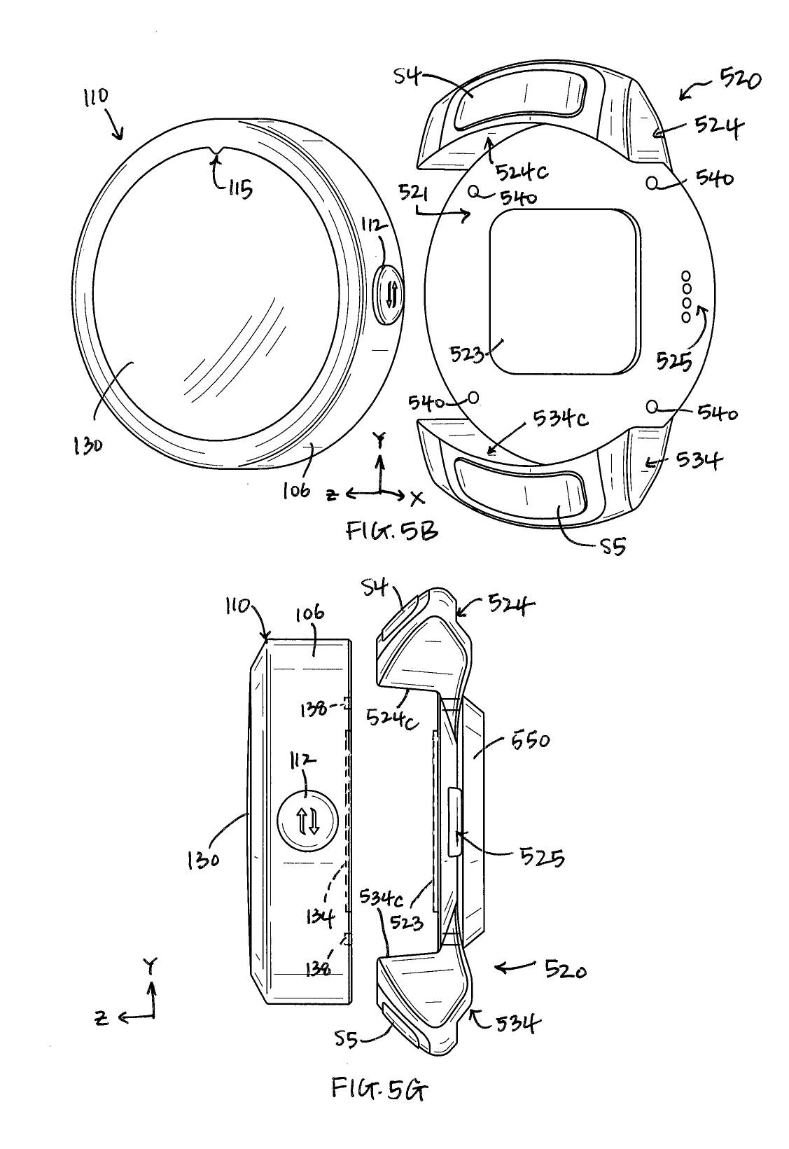

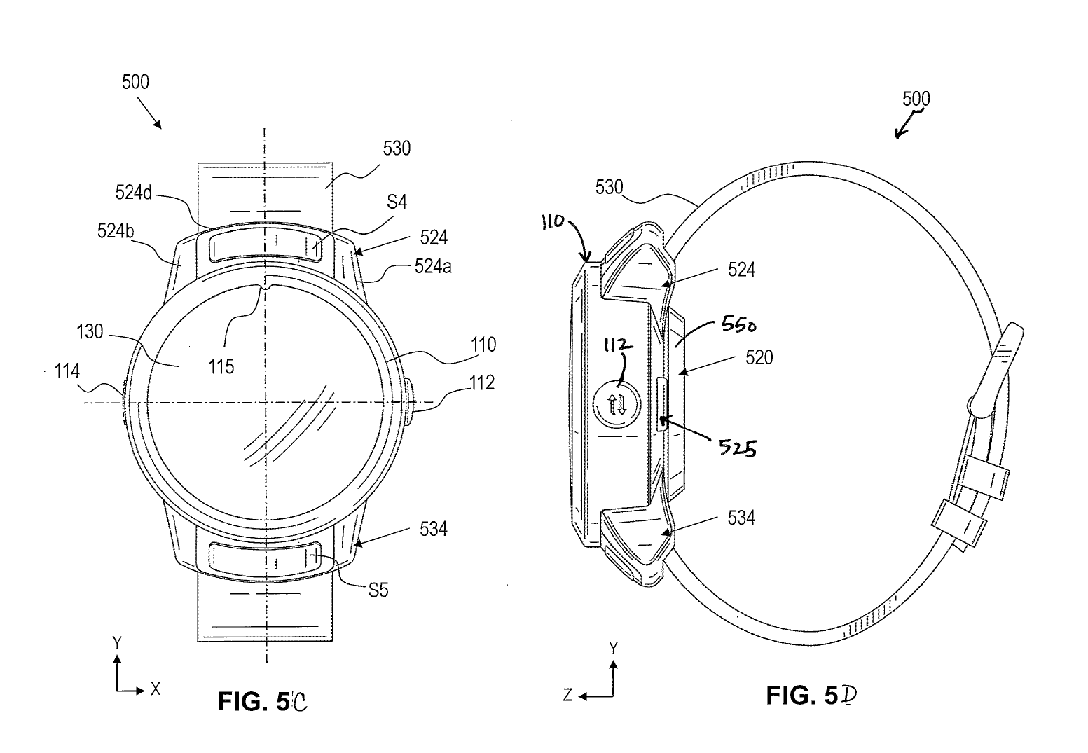

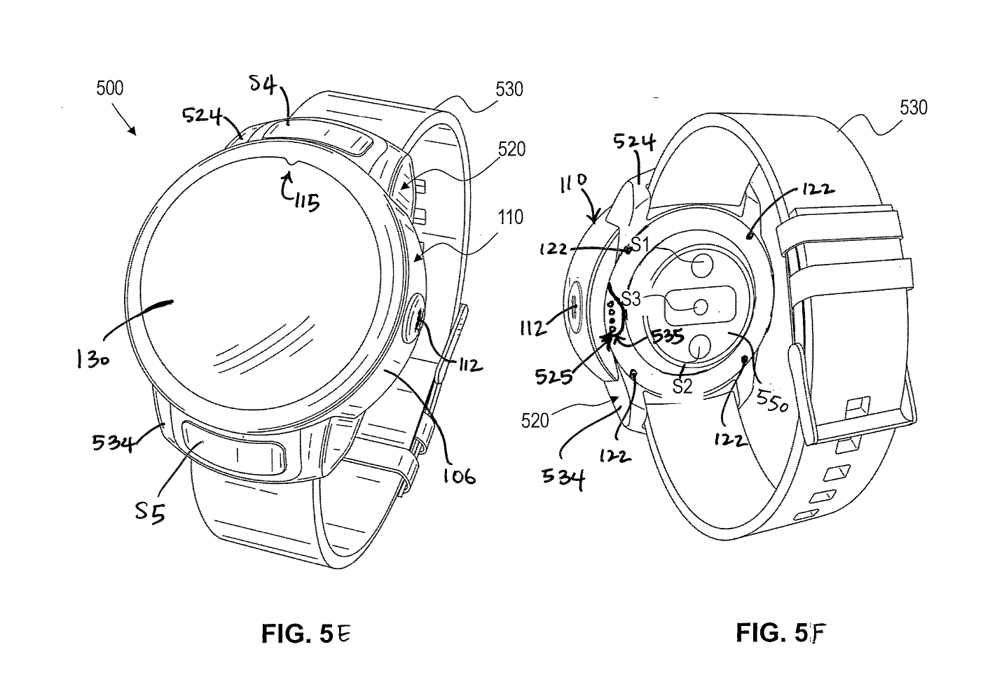

FIG. 5A is an exploded, back side angled view of a fifth interchangeable accessory cover 520 and device case 110 forming a wearable device 500, according to still yet another embodiment of this disclosure. FIGS. 5B-5G illustrate an exploded, front side angled view, a front, a second (right) side, a front perspective, and a back perspective views, respectively, of the wearable device 500. Much like interchangeable accessory cover 120, interchangeable accessory cover 520 is configured to provide accessibility to the electrical connector portion 155 on the back of the device case 110 when attached thereto. In one embodiment, the device case 110 and the accessory cover 520 may be communicably coupled to each other such that the wearable device 500 may establish an electrical connection as well as communication link with an external device when the cover 500 is placed into electrical contact with the external device. Like accessory cover 420, the fifth interchangeable accessory cover 520 may have a portion provided in the form of a plate for placement on the back of the device case. The plate of the accessory cover 520 has an inner surface 521 (see FIG. 5B) and an outer surface 522 (see FIG. 5A). Pairs of lugs that are part of watch lug portions 524 and 534 (described later below) are connected or attached to the plate of the cover 520. In an embodiment, the plate itself, its inner surface 521, and/or outer surface 522 may be similarly shaped to the housing of device case 110—or at least its back plate 132—for example, both have a substantially circular shape or any other shape (e.g., square, rectangular, oval, etc.) that correspond to each other. In one embodiment, the plate associated with cover 520 is substantially disc shaped.

In addition, as shown in FIG. 5B, for example, it should be understood that the interchangeable accessory cover 520 may include a projection 523, that is similar to projection 123, for alignment with a recess (shown in phantom in FIG. 5G, but not shown in FIG. 5A), similar to recess 134, on a back portion of the device case 110, in accordance with an embodiment. Furthermore, the interchangeable accessory cover 520 may also include one or more holes 540 that may be aligned with the receiving holes 138 on the back plate 132 of the device case 110 in a similar manner as described with respect to holes 128 of accessory cover 120. The holes 540 may be configured to receive screws 122 (see FIG. 5F) therethrough that are screwed into and secured onto threads of the receiving holes 138 to secure the interchangeable accessory cover 520 to the device case 110. Of course, the use of holes and screws as the removable attachment mechanism for interchangeable accessory cover 520 is not intended to be limiting, and any other mechanism including those described in detail previously with regards to FIGS. 6A through 10B may be used.

As such, it should be understood that the fifth interchangeable accessory cover 520 may be used with the device case 110, as well as to replace any other interchangeable accessory cover 120, 220, 320, and/or 420, or vice-versa. In this case, the accessory associated with the device case 110 may be changed such that it forms the wearable device 500 for placement around a user's wrist.

An electrical connector portion or electrical contact portion 525, which is equivalent to the previously described electrical contact portion 125 and its embodiments, is disposed on the outer surface 522 of the accessory cover 520. As shown in FIGS. 5A, 5B, and 5F, for example, in one embodiment, the interchangeable accessory cover 520 has its own an electrical contact portion 525 that has one or more electrical contact points. The electrical contact portion 525 may be provided in an area 535 on the accessory cover 520 that may be configured for alignment relative to a location of the electrical connector portion 155, in accordance with an embodiment. In one embodiment, the electrical contact points extend between the front portion or inner surface 521 and the back portion or outer surface 522, such that the electrical contact points are accessible via the outer surface 522/back portion when the interchangeable accessory cover 520 is attached to the device case 110. In another embodiment, holes are provided for receipt of wires therein. Since the features relating to electrical contact portion 525 are similar to the electrical contact portion 125 (e.g., use of wires, four electrical wire contact points, functions (charging, data transfer), etc.) which are described in detail above with respect to FIGS. 1A-1J, they are not all necessarily repeated here. Nonetheless, it should be understood that the accessory cover 520 may include any number of features described with reference to electrical contact portion 125.

The electrical contact points of the electrical contact portion 525 of the accessory cover 520 may be configured for alignment with the wires of the electrical connector portion 155 on the device case 110 such that they are in electrical communication. In one embodiment, part of the electrical contact portion 525 may be placed in direct contact with the electrical connector portion 155. Accordingly, an electrical connection between the processor 150 and the external device may be established using the electrical contact points of electrical contact portion 525. Measurements collected by the measurement device(s) 140 or sensor(s) and processed by the processor 150 may be communicated to the external device via the interchangeable accessory cover 520. In addition, data or information may be communicated to the processor 150 and/or electrical components of the wearable device 400 from the external device via using the electrical contact portion 525 of the accessory cover 520.

Of course, it should also be noted that in yet another embodiment, instead of providing electrical contact portion 525, the accessory cover 520 may include an opening, a hole, a cutout portion, or a shape for allowing access to the electrical connector portion 155 of the device case 110 when the cover 520 is attached thereto, as previously described with reference to cover 120.

Watch lug portions 524 and 534 are of generally similar construction and configured to receive and secure a band 520 (shown in FIGS. 5C-5F) in order to facilitate a user wearing of the wearable device 500 on a wrist, an arm, and/or other body parts. In an embodiment, the lug portions 524 and 534 are located diametrically opposite to each other. Each watch lug portion 524 and 534 has a pair of lugs 524a, 524b and 534a, 534b, respectively, which extend or project from the plate of the accessory cover 520. In one embodiment, the lugs 524a, 524b and 534a, 534b are accessible via the back of the accessory cover 520.

In accordance with one embodiment, watch lug portions 524 and 534 are similar to watch lug portions 424 and 434 of the fourth interchangeable accessory cover 420. In another embodiment, each of the watch lug portions 524 and 534 include bridge portions between their lugs and a sensor. Such an example is further described below.

In the illustrated embodiment, the watch lug portions 524 and 534 (and thus their pairs of lugs) extend both in the Y-direction (or vertically) and the Z-direction (or axially), when viewed in the X-direction (see FIG. 5G). Each pair of lugs 524a, 524b and 534a, 534b may be connected by a bridge portion 524d and 534d, respectively, as shown in FIG. 5A. Generally, the watch lug portions 524 and 534 extend outward (e.g., in a vertical direction or Y-direction) from the plate or outer surface 522, while the bridge portions 524d and 534d extend in generally horizontal direction that is parallel to the horizontal centerline (see FIG. 5C) of the wearable device 500. In an embodiment, the front surface of each base portion 424d and 434d may be positioned such that it extends from the edge 106 of the device case 110 when assembled thereto.

As shown in FIG. 5B, in accordance with an embodiment, each watch lug portion 524 and 534 may include a curved surface 524c and 534c, respectively, that extends forwardly from the inner surface 521. The curved surfaces 524c and 534c each have an arc or a curvature that cooperates or confirms with the sides or edge 106 of the device case 110. Accordingly, when the accessory cover 520 is attached to the device case 110, the curved surfaces 524c and 534c may be provided along or against the perimeter or edge 106 of the device case 110.

Generally each lug 524a, 524b and 534a, 534b may extend in a generally perpendicular direction (e.g., in a vertical direction or Y-direction) relative to edge 106, for example, in the wearable device 500. The pair of lugs 524a and 524b at the top end of the accessory cover 520, as shown in FIG. 5A, are spaced from one another, e.g., at either side (right and left). A space between the pair of lugs 524a and 524b is configured for receipt of an end of a watch band 530, for example. Specifically, each lug 524a and 524b has a receiving hole therein that is configured to receive an end or a portion of a pin 526 therein. The receiving holes are provided in the internal surfaces of the lugs 524a and 524b (i.e., the surfaces that are facing each other), and are horizontally aligned. The receiving holes may be through holes or blind holes in each of the lugs. In another embodiment, the receiving holes may be provided as dimples that are horizontally aligned with each other. Similarly, the pair of lugs 534a and 534b positioned at the bottom end of the accessory cover 520 such that the lugs 534a and 534b are spaced from one another, e.g., at either side (right and left) and configured, for receipt of another end of the band therebetween, for example. Lugs 534a and 534b also have receiving holes or dimples provided therein as described above (and thus not repeated here) for placement of a pin 536 therein.

In an embodiment, gaps 527 and 537 (shown in FIG. 5A) are provided between the pins 526 and 536 and an edge surface on the watch lug portions 524 and 534, respectively, to accommodate the ends of the band. Gaps 528 and 538 may also be provided between the pins 526 and 536 and a surface of the bridge portions 524d and 534d.

In accordance with one embodiment, to attach a band 530, the pin 526 may be placed through a through hole (not shown) on one end of the band 530, and its ends may be inserted and secured in the receiving holes of the lugs 524a and 524b. Pin 536 may be placed through a through hole (not shown) on the other end of the band 530, and its ends may be inserted and secured in the receiving holes of the lugs 534a and 534b. In one embodiment, the pins 526 and 536 may each be a spring loaded shaft that is configured to compress in some manner, to allow ends of the pins 526 and 536 to be inserted within the receiving holes/dimples before expanding to its original length.

In another embodiment, the pins 526 and 536 may be secured within and between the lugs 524a, 524b and 534a, 534b. In such a design, the pins 526 and 536 support to a band 530 whose respective ends are fed through gaps 527, 528 and 537, 538 in order to wrap each end portion of the band around the respective pin 526 or 536, for example.

In accordance with one embodiment, interchangeable accessory cover 520 further includes sensors and/or measurements devices associated therewith, for sensing and collecting measurements and data from the user and sending it to processor 150. For example, as shown in FIG. 5A, one or more sensors, including portions S1, S2, and S3, are disposed on the outer surface 522 of the accessory cover 520, in accordance with an embodiment. In an embodiment, shown in FIG. 5C, sensors S4 and S5 are provided on lug portions 524 and 525. Each of the sensors is configured to communicate with the processor 150 via the electrical contact portion 525 and electrical connector portion 155, for example, in one embodiment. In another embodiment, one or more of the sensors may be configured to communicate wirelessly with the processing circuitry 150 of the device case 110. Each of the sensors may be a physiological sensor such as a heart rate sensor, a blood sugar level sensor, a temperature sensor, a skin conductivity sensor, an electrocardiograph sensor, or other biological or physiological sensors.

In one embodiment, portions S1-S3 are part of a heart rate sensor that is provided within the accessory cover 520, while sensors S4 and S5 are electrodes used to measure skin conductivity and electricity (for example, ECG).

In an embodiment, one or more sensors are contained within the accessory cover 520. For example, the accessory cover 520 may include a housing to contain a sensor or sensor related parts therein. The housing may be perceived as a protuberance 550 (e.g., see FIGS. 5D and 5G) extending from the outer surface 522 of the accessory cover 520. Portions S1, S2, and S3 are provided on a back surface of the protuberance 550 or house and may include windows for optical sensors that may be used for heart rate and/or blood pressure monitoring, for example. Light sources, e.g., LEDs (e.g., red and green lights), and/or photodetectors may be contained in the protuberance 550 and used sensing devices. A printed circuit board (PCB) may also be provided in the housing or protuberance 550 that is associated with sensor portions such as the light source(s) and photodetector. When the protuberance 550 and thus sensor portions S1, S2, and S3 are placed against a user's skin (e.g., on the wrist or arm area), the light source(s), photodetector, and PCB may be used to detect a user's heart rate, for example. A number of additional health related measurements or data may be calculated using the sensed data. Such functions and parts associated with these type of heart rate sensors and monitors are generally known by one of ordinary skill in the art, and thus not further explained here.

In accordance with an embodiment, communication between any PCB or sensing device contained within the housing or protuberance 550 and processor 150 of device case 110 is established via electrical contact portion 125 and electrical connector 155.

FIGS. 5B and 5C best illustrate an example of sensors S4 and S5 provided on a front of each of the lug portions 524 and 534 of the accessory device 520. Sensors S4 and S5 are generally rectangular in shape, but may be sized or altered in shape based on the size and shape of the lug portions 524 and 534. The sensors S4 and S5 may be electrically connected with a PCB contained with the housing of accessory cover 520 (e.g., in protrusion 550), in accordance with an embodiment. Sensors S4 and S5 may communicate with the PCB or with processor 150 of the device case 110. As noted above, sensors S4 and S5 may be electrodes used to measure skin conductivity and electricity. In accordance with an embodiment, communication between sensors S4 and S5 and processor 150 of device case 110 may be established via electrical contact portion 125 and electrical connector 155.

In an embodiment, the lug portions 524 and 534 may be integrally formed with the plate and/or its outer surface 522 (e.g., via molding) or attached thereto (e.g., by molding, soldering, or adhesive) to from an integral structure. However, the materials and manufacturing methods used to form the interchangeable accessory cover 520 are not limited. The accessory cover 520 may be made of similar material as the device case 110, for example, aluminum, steel, gold, silver, other metallic material, plastic, and formed using a similar manufacturing process, e.g., injection molding or casting. In an embodiment, the accessory cover 520 may be made of a different material than the housing of the device case 110. Also, the accessory cover 520 may be formed from multiple materials. Additionally, any type of band 530 may be used with wearable device 500. The band 530 may be formed from leather, plastic, or cloth materials, for example.

As noted throughout, any of the alternate designs and/or mechanical mechanisms for attaching device case 110 and an interchangeable accessory cover may be provided on any one or more of the other exemplary covers 120, 220, 320, 420, and 520 described herein, such that they may be attached to the device case 110 form the wearable device.

In an embodiment, the mechanical mechanisms may vary between each type of accessory cover. For example, in one embodiment, two or more interchangeable accessory covers may be provided with device case 110. In one embodiment, the at least two interchangeable accessory covers have different types of mechanical mechanisms for connecting the respective accessory cover to the device case 110 (e.g., one has screws and holes, while the other has a snap-fit connection). In another embodiment, the at least two interchangeable accessory covers each have a mechanical mechanism for connecting the respective accessory cover to the device case, and the mechanical mechanisms are of the same type (e.g., screws and holes).

More than one interchangeable cover may be provided with the device case 110, for sale as a system. In one embodiment, such a system may include a device case, a first type of interchangeable accessory cover, and a second type of interchangeable accessory cover, wherein the second interchangeable accessory cover has a different configuration than the first interchangeable accessory cover, but both are configured to provide accessibility to the electrical connector portion on the back of the device case when attached thereto. For example, one of the accessory cover may be similar to cover 120, while the other includes a necklace attachment portion 202, a clip 324, pairs of watch lugs to connect with a band or strap, and/or one or more physiological sensors. In one embodiment, a third interchangeable accessory cover adapted to be removably attached to the device case may be included in the system that has a different configuration than both the first and second types of covers. In another embodiment, the wearable device may be provided or sold with one type of accessory cover, and other types of accessory covers may be obtained or purchased.

In an embodiment, any one or more (or all) of the herein disclosed interchangeable accessory covers 120, 220, 320, 420, and/or 520 may include a cutout portion providing access to the electrical connectors 125 of the device case. In accordance with an embodiment, the cutout portion may be provided in or on an accessory cover without its corresponding electrical contact portion (125, 225, 325, 425, and/or 525). The cutout portion of the interchangeable accessory cover may be at a location corresponding to the electrical connector portion 155 of the device case 110. For example, the area 135, 235, 335, 435, and/or 535 for the electrical contact portions may instead be a cutout portion. Accordingly, in an embodiment, instead of providing the accessory cover with its electrical contacts, the cutout portion may be aligned with respect to the electrical connector portion 155 to provide accessibility to the electrical connector portion 155 on the back of the device case 110 when attached thereto. For example, in one embodiment, the cutout portion may be located near or at the perimeter or edge of the interchangeable accessory cover. In an embodiment, the cutout portion may cut through the perimeter or edge of the interchangeable accessory cover (e.g., extend from the edge and into the outer surface in an axial direction, e.g., in the X direction). In another embodiment the cutout portion, for example, includes a hole or opening that may be located near the perimeter or edge of the cover, but does not cut through the perimeter or edge (i.e., the edge defines the hole or opening). In an embodiment, the interchangeable accessory cover may be shaped such that when its body is positioned on or attached to the device case 110, e.g., positioned on the back of the device case 110, the electrical connectors 155 may be accessed. For example, instead of a plate or disc, the interchangeable accessory cover may have a body that has concave edges or curved edges that, when aligned with the back of the device case 110, provide access to back plate 132 and connector portion 155. In one embodiment, the body of the interchangeable accessory cover has a smaller surface area than that of the back 132 of the device case 110 to provide access to the electrical connector portion 155. In an embodiment, the interchangeable accessory cover may be placed over the device case and the attachment may be a C-shaped snap clamp that fits over the interchangeable accessory cover and snaps at the perimeter of the device case, thus securing the interchangeable accessory cover to the device case, while providing access to the electrical connectors of the interchangeable accessory cover and/or the device case.

It may be understood by a person skilled in the art that the term wearable device 100 may be used interchangeably with wearable device 200, 300, 400, 500, etc. depending on the type of the interchangeable accessory cover attached. Furthermore, the shape of the wearable device 100 is presented as being circular in the Figures by way of an example only. This disclosure should not be limited by the shape of the wearable device 100, device case 110, and/or interchangeable accessory covers as depicted in the Figures. In some embodiments, the wearable device 100 and its parts may be rectangular, square, triangular, ovular, or other aesthetically pleasing shapes. Furthermore, one or more interchangeable accessory covers may be modified to adopt to the desired shape of the wearable device 100.

While the principles of the disclosure have been made clear in the illustrative embodiments set forth above, it will be apparent to those skilled in the art that various modifications may be made to the structure, arrangement, proportion, elements, materials, and components used in the practice of the disclosure.

It will thus be seen that the features of this disclosure have been fully and effectively accomplished. It will be realized, however, that the foregoing preferred specific embodiments have been shown and described for the purpose of illustrating the functional and structural principles of this disclosure and are subject to change without departure from such principles. Therefore, this disclosure includes all modifications encompassed within the spirit and scope of the following claims.

1. A wearable device comprising:
a device case comprising:
at least one measurement device and a processor for processing measurements collected by the measurement device that are enclosed within the case;
a display or screen on a front of the device case for displaying data corresponding to measurements measured by the at least one measurement device;
a first button provided on one side of the case, the first button comprising a raised, tactile portion thereon, and a second button provided on an opposite side of the case relative to the first button, the first and second buttons being configured to implement a function or an action via the processor when pressed by a user;
an electrical connector portion provided on a back of the device case that is used to establish an electrical connection between the processor and an external device such that the processor and the external device are configured for communication and measurements that are collected by the at least one measurement device and processed by the processor are communicated to the external device; and
an interchangeable accessory cover removably attached to the device case, wherein the accessory cover is configured to provide accessibility to the electrical connector portion on the back of the device case when attached thereto,
wherein the device case further comprises a pointed portion extending from a front edge thereof and over or into the display or screen on the front of the device case, acting as an orientation indicator indicating a position for viewing the display or screen, and wherein an edge of the display or screen is formed such that it includes a corresponding or complimentary shaped indentation or cutout for receiving at least a portion of the pointed portion therein.

2. The wearable device according to claim 1, wherein the interchangeable accessory cover is configured to cover at least a portion of the back of the device case when attached to the device case.

3. The wearable device according to claim 1, wherein the interchangeable accessory cover comprises a front portion, a back portion, and a plurality of electrical contact points, the front portion being configured for placement against a portion of the device case and the electrical contact points extending between the front portion and the back portion such that the electrical contact points are accessible via the back portion upon attachment of the interchangeable accessory cover to the device case; and
wherein the electrical contact points of the interchangeable accessory cover are configured to communicate with the electrical connector portion of the device case, such that the electrical connection between the processor and the external device is established using the electrical contact points that are accessible via the back portion of the interchangeable accessory cover.

4. The wearable device according to claim 1, wherein the interchangeable accessory cover and the device case are attached to one another using one or more screws.

5. The wearable device according to claim 1, wherein the interchangeable accessory cover and the device case comprise complimentary portions for attaching the interchangeable accessory cover and the device case together.

6. The wearable device according to claim 1, wherein at least one of the interchangeable accessory cover and/or the device case comprises a snap fit connector for attaching the device case and interchangeable accessory cover together.

7. The wearable device according to claim 1, wherein the interchangeable accessory cover comprises a necklace attachment portion extending outwardly from an edge of the interchangeable accessory cover, the necklace attachment portion having a hole therethrough.

8. The wearable device according to claim 1, wherein the interchangeable accessory cover comprises a clip having a resilient spring arm portion configured to receive and secure a piece of clothing between the spring arm portion and a surface of the interchangeable accessory cover.

9. The wearable device according to claim 1, wherein the interchangeable accessory cover comprises pairs of watch lugs, the pairs being provided on opposite sides of the cover and projecting outwards from the device case, wherein each lug of the pairs has a receiving hole therein that is configured to receive a portion of a pin used for attaching a watch band or watch strap.

10. The wearable device according to claim 9, wherein the interchangeable accessory cover further comprises one or more physiological sensors.

11. The wearable device according to claim 10, wherein the one or more physiological sensors is selected from the group consisting of: a heart rate sensor, a skin conductivity sensor, a blood sugar level sensor, a temperature sensor, and an electrocardiograph sensor.

12. The wearable device according to claim 1, wherein the device case further comprises an audio device therein and wherein speaker holes are provided on the device case for emitting sound from the audio device.

13. (canceled)

14. The wearable device according to claim 1, wherein the device case comprises a housing that is circular in shape and wherein an outer edge of the interchangeable accessory cover is circular in shape.

15. A system comprising:
a wearable device comprising:
a device case comprising:
at least one measurement device and a processor for processing measurements collected by the measurement device that are enclosed within the case;
a display or screen on a front of the device case for displaying data corresponding to measurements measured by the at least one measurement device;
a first button provided on one side of the device case, the first button comprising a raised, tactile portion thereon, and a second button provided on an opposite side of the device case, relative to the first button, the first and second buttons being configured to implement a function or an action via the processor when pressed by a user; and
an electrical connector portion provided on a back of the device case that is used to establish an electrical connection between the processor and an external device such that the processor and the external device are configured for communication and measurements that are collected by the at least one measurement device and processed by the processor are communicated to the external device;
a first interchangeable accessory cover adapted to be removably attached to the device case, wherein the first interchangeable accessory cover is configured to provide accessibility to the electrical connector portion on the back of the device case when attached thereto; and
a second interchangeable accessory cover also adapted to be removably attached to the device case, the second interchangeable accessory cover having a different configuration than the first interchangeable accessory cover, wherein the second interchangeable accessory cover is also configured to provide accessibility to the electrical connector portion on the back of the device case when attached thereto.

16. The system according to claim 15, wherein each of the first and second interchangeable accessory covers are configured to cover at least a portion of the back of the device case when either is attached to the device case.

17. The system according to claim 15, wherein each of the first and second interchangeable accessory covers comprises a front portion, a back portion, and a plurality of electrical contact points, the front portion being configured for placement against a portion of the device case and the electrical contact points extending between the front portion to the back portion such that the electrical contact points are accessible via the back portion upon attachment of the interchangeable accessory cover to the device case; and
wherein the electrical contact points of each of the first and second interchangeable accessory covers are configured to communicate with the electrical connector portion of the device case, such that the electrical connection between the processor and the external device is established using the electrical contact points that are accessible via the back portion of the respective interchangeable accessory cover.

18. The system according to claim 15, wherein the each of the first and second interchangeable accessory covers have a mechanical mechanism for connecting the respective accessory cover to the device case, and wherein the mechanical mechanisms are of the same type.

19. The system according to claim 15, wherein the each of the first and second interchangeable accessory covers have a mechanical mechanism for connecting the respective accessory cover to the device case, and wherein the mechanical mechanisms are of a different type.

20. The system according to claim 15, wherein one of the first and second interchangeable accessory covers includes a necklace attachment portion extending outwardly from an edge of the interchangeable accessory cover, the necklace attachment portion having a hole therethrough.

21. The system according to claim 15, wherein one of the first and second interchangeable accessory covers includes a clip having a resilient spring arm portion configured to receive and secure a piece of clothing between the spring arm portion and a surface of the interchangeable accessory.

22. The system according to claim 15, wherein one of the first and second interchangeable accessory covers includes pairs of watch lugs, the pairs being provided on opposite sides of the cover and projecting outwards from the device case, wherein each lug of the pairs has a receiving hole therein that is configured to receive a portion of a pin used for attaching a watch band or watch strap.

23. The system according to claim 22, wherein one of the first and second interchangeable accessory covers includes one or more physiological sensors selected from the group consisting of: a heart rate sensor, a skin conductivity sensor, a blood sugar level sensor, a temperature sensor, and an electrocardiograph sensor.

24. The system according to claim 15, further comprising a third interchangeable accessory cover adapted to be removably attached to the device case, wherein the third interchangeable accessory cover is configured to provide accessibility to the electrical connector portion on the back of the device case when attached thereto, the third interchangeable accessory cover having a different configuration than both the first and second interchangeable accessory covers.

25. An interchangeable accessory cover for removable attachment to a wearable device, the wearable device comprising: a housing that contains at least one measurement device and a processor for processing measurements collected by the measurement device, and a display or screen on a front of the housing for displaying data corresponding to measurements measured by the at least one measurement device; the accessory cover comprising:
a front portion and a back portion,
a beveled edge extending between the front portion and the back portion, and
an electrical connector portion with electrical contact points that are accessible via the back portion upon attachment to the wearable device,
wherein the electrical connector portion is configured to establish an electrical connection between the processor of the wearable device and an external device such that the processor and the external device are configured for communication and measurements that are collected by the at least one measurement device and processed by the processor are communicated to the external device via the interchangeable accessory cover.

26. The interchangeable accessory cover according to claim 25, wherein the interchangeable accessory cover is configured to cover at least a portion of a back of the wearable device when attached thereto.

27. The interchangeable accessory cover according to claim 25, wherein the electrical contact points extend between the front portion to the back portion; and
wherein the electrical contact points on the front portion of the interchangeable accessory cover are configured for alignment with a corresponding electrical connector on the wearable device, such that the electrical connection between the processor and the external device is established and accessible via the back portion of the interchangeable accessory cover.

28. The interchangeable accessory cover according to claim 25, wherein the interchangeable accessory cover has holes therein for receipt of one or more screws for attachment of the cover to the wearable device.

29. The interchangeable accessory cover according to claim 25, further comprising a threaded portion or screw portion configured for attachment to a complimentary threaded or screw portion of the wearable device.

30. The interchangeable accessory cover according to claim 25, further comprising a snap fit connector for attaching to the wearable device.

31. The interchangeable accessory cover according to claim 25, further comprising a necklace attachment portion extending outwardly from an edge of the interchangeable accessory cover, the necklace attachment portion having a hole therethrough.

32. The interchangeable accessory cover according to claim 25, further comprising a clip having a resilient spring arm portion configured to receive and secure a piece of clothing between the spring arm portion and a surface of the interchangeable accessory cover.

33. The interchangeable accessory cover according to claim 25, further comprising pairs of watch lugs, the pairs being provided on opposite sides of the cover and configured to project outwardly from the wearable device upon attachment thereto, wherein each lug of the pairs has a receiving hole therein that is configured to receive a portion of a pin used for attaching a watch band or watch strap.

34. The interchangeable accessory cover according to claim 33, further comprising one or more physiological sensors selected from the group consisting of: a heart rate sensor, a skin conductivity sensor, a blood sugar level sensor, a temperature sensor, and an electrocardiograph sensor.

35. The interchangeable accessory cover according to claim 25, wherein the front and back portions are similar to or the same shape as a back of the housing of the wearable device, and wherein the front portion is configured for placement against the back of the wearable device.
